(19) United States
(12) Patent Application Publication
(10) Pub. No.: US 2026/0151601 A1
(43) Pub. Date: Jun. 4, 2026

(54) STENT-LIKE CATHETER FOR ISOLATING A REGION IN A HOLLOW ORGAN OF A MAMMAL, AND SYSTEM BASED ON THE CATHETER

(71) Applicant: PANDICA LTD, London (GB)

(72) Inventors: Aleksei Arievich KASHINTSEV, Saint-Petersburg (RU); Vitali Yurievich PROUTSKI, Saint-Petersburg (RU); Sergey Vladimirovich ANISIMOV, Saint-Petersburg (RU); Oleg Konstantinovich GRANSTREM, Saint-Petersburg (RU)

(21) Appl. No.: 18/706,300

(22) PCT Filed: Nov. 1, 2021
(86) PCT No.: PCT/RU2021/000477

§ 371 Date: Apr. 30, 2024

(51) Int. Cl. A61M 25/10; A61B 10/00; A61B 17/00; A61B 17/12; A61M 25/00

(52) U.S. Cl. CPC A61M 25/1011; A61B 10/0045; A61B 17/12036; A61B 17/12045; A61B 17/12136; A61M 25/0043; A61B 2010/0083; A61B 2017/00544; A61B 2017/00557; A61B 2017/00561; A61M 2025/1052; A61M 2025/1084; A61M 2025/1095; A61M 2202/0014; A61M 2202/02; A61M 2202/04; A61M 2205/3344

(57) ABSTRACT

Catheter devices, systems, and methods are disclosed herein related to a catheter for isolating a region in a hollow organ of a mammal. The catheter can include an elongate body including a proximal end, a distal end, and a central lumen. The proximal end may define a first opening and the distal end may define a second opening. The catheter may include a plurality of proximal extensions coupled to the proximal end of the elongate body, the plurality of proximal extensions including a plurality of individual channels continuous with the elongate body. The catheter can include two balloons configured to be inflated to isolate an interior of the hollow organ, and a functional channel designed to allow a negative pressure to be produced in the isolated interior to take a fluid or gaseous medium therefrom or a liquid or gaseous medium to be supplied into the isolated interior.

### FIELD OF THE INVENTION

The present disclosure relates to medicine, and more particularly to medical devices used to diagnose and/or treat inflammatory, autoimmune, infectious, benign and/or malignant diseases that occur in hollow organs and organs that are connected to the hollow organs. More particularly, the present disclosure may be used to diagnose and/or treat particular diseases of blood vessels, pancreas, bile ducts, gallbladder, liver, gastrointestinal tract, stomach, and duodenum, respiratory tracts, identify and treat defects within a mammal hollow organ such as fistulas, strictures, aneurismal and diverticular dilatations, and other diseases of mammal hollow organs and organs connected to the hollow organs, and isolate a region of a hollow organ to enable surgical manipulation and/or sampling of the isolated region of the hollow organ or provision of medicinal substances into the isolated region of the hollow organ.

### BACKGROUND

Known in the art are various devices and appliances used to diagnose and/or treat inflammatory, autoimmune, infectious, benign and/or malignant diseases that occur in hollow organs and organs connected to hollow organs of a mammal, in particular diseases of pancreas, bile duct diseases, liver diseases, gallbladder diseases and also gastrointestinal diseases, gastrointestinal wall defects, diseases and defects of blood vessels, injuries of the wall of a hollow organ, aneurismal and diverticular dilatation, strictures of hollow organs, bleeding of hollow organs, bleeding into hollow organs and other diseases of hollow organs and organs that are connected to hollow organs.

However, known catheters have several drawbacks. For example, there exist catheters for isolating an area within a hollow organ (e.g., through the inflation of isolating balloons) for taking or delivering a biological fluid specific to the hollow organ. But currently known catheters have an issue in that it cannot be inserted into a lumen of a hollow organ for a long time due to the lack of physiological connectivity between the hollow organ sections adjoining the hollow organ region isolated by inflation balloons. That is, once the catheter is positioned in place, there is no way for biological fluids to pass around the isolated zone.

Another known drawback of existing catheter systems is that the functional opening (e.g., sampling port) that opens into the isolated area of the hollow organ can become blocked or occluded by tissue of the hollow organ during sampling and/or aspiration of the isolated area.

Accordingly, a catheter for isolating a region in a hollow organ of a mammal that enables biological fluids to pass around the isolated zone is needed. Consequently, a technical problem that is solved by the present disclosure is the development of a catheter for isolating a region in a hollow organ that can be maintained in the lumen of a hollow organ for long periods of time while maintain the function (e.g., bypassing of biological fluids) of the hollow organ. Another technical problem that is solved by the present disclosure is the development of a catheter that facilitates bidirectional bypassing of fluids around the isolated area, allowing the catheter to be inserted into a patient in any orientation (e.g., proximal end towards the direction of fluid flow or away from the direction of fluid flow) while allowing for the flow of fluids through both a proximal and distal end of the catheter.

According to guidelines for diagnosing and treating a pancreatic cancer, main techniques allowing for a histological type of neoplasm to be confirmed are fine-needle percutaneous core-biopsy, fine-needle aspiration biopsy, cytological analysis of an epithelial scraping (also referred to in the art as a brush-biopsy), diagnostic laparoscopy with a biopsy, and cytological analysis of washings obtained from an abdominal cavity during laparoscopy or laparotomy (Pancreatic adenocarcinoma guidelines. National Cancer Comprehensive Network. 2021 Version 1.2021). These officially recommended biopsy techniques are based on different principles, and therefore they cannot be regarded as the closest prior art for the present invention.

In particular, known in the art is an ultrasonic method for studying pancreas (Minko B. A. Complex beam diagnostics of pancreas diseases. In: Minko A. B., Pruchansky B. C., Korytova L. I.—Complex beam diagnostics of pancreas diseases. Hippokrat, Saint-Petersburg, Russia; 2001; 134 p.; Martínez-Noguera A., Montserrat E., Torrubia S. et al.; Ultrasound of the pancreas: update and controversies. Eur Radiol 2001; 11: 1594-1606). The ultrasonic method is based on the assessment of changes in the reflection and absorption of waves from an organ tissue and allows for gland contours, liver contours and alternations of hyperechoic or hypoechoic zones to be determined. Furthermore, the ultrasonic method allows for determination a Wirsung duct formation and its diameter, a bile duct formation and its diameter, and other formations and their diameters. The main disadvantage of an ultrasonic method is in that examination result depends on a specialist's qualification and an apparatus' resolution. In this case, the sensitivity of the ultrasound examination is 70-80%. If a size of the formation is less than 1.5 cm, the ultrasonic method has strongly reduced effectiveness. In particular, the ultrasonic method does not allow for intraductal neoplasms to be detected, the pancreatic duct to be completely evaluated, a biochemical composition of pancreatic juice and bile to be evaluated, and pancreatic juice and bile to be cytologically examined.

Furthermore, known in the art is a method of endoscopic ultrasound examination based on the same principle as the above-described ultrasonic method (see Ogawa M., Kawaguchi Y., 2011; Iglesias-Garcia J., 2012). An advantage of the endoscopic ultrasound examination over the ultrasonic method is in that biopsy may be performed in close proximity to a site of interest. A convex probe, if any, allows for a suspicious formation to be examined with a fine-needle functional biopsy followed by a cytological examination. Disadvantages of the endoscopic ultrasound examination are the following: necessity to have a highly skilled specialist, necessity to perform anesthetic aid, high costs, and lack of possibility to perform examination in some cases due to some anatomical features. Furthermore, endoscopic ultrasound examination is not suitable for performing cytologic diagnostics of liver and of bile ducts and allows only for a small amount of cytological material to be obtained by performing aspiration, so that it is difficult to interpret the obtained cytological material, causing a high proportion of false-positive results and false-negative results. Furthermore, the endoscopic ultrasound examination does not allow for a qualitative analysis of the composition of bile or pancreatic juice.

Furthermore, known in the art is a fine-needle biopsy, wherein the fine-needle core-biopsy is always used in combination with the above-described ultrasonic method and the above-described endoscopic ultrasound examination and allows for a material for histological examination to be obtained (Hruban R. H., Takaori K., Klimstra D. S. An illustrated consensus on the classification of pancreatic intraepithelial neoplasia and intraductal papillary mucinous neoplasms. Am J Surg Pathol 2004; 28 (8): 977-987). The fine-needle biopsy is a main method used to histologically verify pancreatic diseases. Disadvantages of the fine-needle biopsy are possible complications: bleeding, formation of fistulas, abscesses, dissemination of cancer cells along a puncture channel, and also uninformative, false-positive or false-negative results of histocytological examinations. Furthermore, the fine-needle biopsy is not suitable for diagnosing diseases of the bile ducts and gallbladder and does not allow for the laboratory evaluation of bile or pancreatic juice.

Furthermore, known in the art is a method of spiral computer-assisted tomography with an intravenous contrast (MSCT) (Callery M. R. et al., 2009; Klaub M. et al., 2009). The spiral computer-assisted tomography is based on the computer-assisted processing of thin “slice” tomograms, assessment of the degree of absorption of a contrast agent by the tissue of a gland or tumor, and the measuring of a diameter of the ducts. The spiral computer-assisted tomography allows for the visualization of formations and making a decision on whether an acute or chronic inflammatory change of corresponding organ is observed based on changes in organ densitometric parameters. Disadvantages of the spiral computer-assisted tomography are significant decrease in its sensitivity when the formation's size is less than 1.5 cm, and low sensitivity and specificity when diagnosing intraductal neoplasms. Furthermore, spiral computer-assisted tomography does not allow for a disease to be histologically confirmed and does not allow for pancreatic secretion to be analyzed.

Furthermore, known in the art is an endoscopic retrograde cholangiopancreatography (ERCP) method (Kawaguchi Y., Ogawa M., Omata F. Randomized controlled trial of pancreatic stenting to prevent pancreatitis after endoscopic retrograde cholangiopancreatography. World Journal of Gastroenterology 2012; 18 (14): 1635-1641). In the endoscopic retrograde cholangiopancreatography, focal formations and calculi are revealed in the form of filling defects. Disadvantages of the endoscopic retrograde cholangiopancreatography are the need for anesthetic aid, a number of limitations preventing certain manipulations, and injuries associated with the procedure, potentially causing destructive pancreatitis or acute cholangitis.

Furthermore, known in the art is an endoscopic aspiration method for aspirating pancreatic juice from duodenum interior by stimulating excretory function of pancreas with Chirhostim™, which is a synthetical analogue of secretin (Suenaga M., Sadakari Y., Almario J. A. et al. Using an endoscopic distal cap to collect pancreatic fluid from the ampulla. Gastrointest Endosc 2017; 86 (6): 1152-1156; Kanda M., Sadakari Y., Borges M. et al. Mutant TP53 in Duodenal samples of pancreatic juice from patients with pancreatic cancer or high-grade dysplasia. Clin Gastroenterol Hepatol 2013; 11 (6): 719-730). The endoscopic aspiration method is used for sampling a material followed by cytological examination and/or molecular genetic analysis thereof. Disadvantages of the endoscopic aspiration method are as follows: lack of public acceptance, and limitation of the amount of sampled pancreatic secretion by duration of drug's action by short duration of the procedure. It is of note that composition of pancreatic juice may change when secretin or its analogues is administered, so that the sampled pancreatic secretion in this case does not have its natural composition, and therefore it is impossible to evaluate the real composition and secretion clearance of pancreatic juice since the function is stimulated by the drug. The endoscopic aspiration method is also not suitable for collecting and analyzing bile.

Furthermore, known in the art is a magnetic resonance imaging (MRI) method used for determining neoplasms and inflammatory changes of pancreas and/or bile ducts (Akisik M F, Sandrasegaran K., Aisen A A. Dynamic secretin enhanced MR cholangiopancreatography. RadioGraphics 2006; 26: 665-677). The magnetic resonance imaging is a non-invasive method and provides an increased sensitivity and specificity when used in combination with radiographic opacification or stimulation of excretory function of pancreas. The disadvantage of the magnetic resonance imaging is the impossibility of evaluating a composition and clearance of bile or pancreatic juice and performing cytological examination thereof.

Furthermore, known in the art is a method of performing a nasopancreatic or nasobiliary drainage followed by the isolated sampling of a substrate (Handa K., Minami T., Shimizu A. et al. Roles of ERCP in the Early Diagnosis of Pancreatic Cancer. Diagnostics 2019; 9 (1): 30; Budzinsky S. A., Shapovalyants S. G., Fedorov E. D., Shabrin A. V. Endoscopic transpapillary pancreatic stenting in the treatment of pancreatic fistulas (with a commentary by A. G. Krieger). N. I. Pirogov Russian Journal of Surgery 2017; (2): 32-44). The nasopancreatic or nasobiliary drainage is used both for treating inflammatory pancreatic diseases and for performing their cytological verification, wherein the nasopancreatic or nasobiliary drainage is suitable for compositional analysis of biofluids. Furthermore, the nasopancreatic or nasobiliary drainage is a single method allowing for pancreatic juice and bile to be independently sampled. A disadvantage of the nasopancreatic or nasobiliary drainage is the complexity of implementation. For the first time, the use of the nasopancreatic or nasobiliary drainage was described in 1980, however the nasopancreatic or nasobiliary drainage is not a routine method and used only in highly specialized institutions, in particular for solving a limited range of tasks, primarily for treating acute pancreatitis. In some cases, particularly due to the presence of some anatomical features, the nasopancreatic or nasobiliary drainage may not be implemented and may cause various complications such as pancreatitis, cholangitis and bleeding.

Furthermore, known in the art is a two-channel Dreiling tube used for performing duodenal intubation (Stevens T., Conwell D L, Zuccaro G. et al. A prospective crossover study comparing secretin-stimulated endoscopic and Dreiling tube pancreatic function testing in patients evaluated for chronic pancreatitis. Gastrointestinal Endoscopy 2008; 67 (3): 458-466; Pollack B J, Grendell J H. Where have all the Dreiling tubes gone? Am J Gastroenterol 2006; 101 (2): 356-359). The Dreiling tube allows for a non-invasive sampling of pancreatic juice and bile followed by a biochemical test and/or cytological examination thereof and by determination of daily clearance. A disadvantage of the Dreiling tube is that it does not allow for the selective sampling of the content of duodenum, leading to the retrograde sampling of intestinal contents. The passive nature of sampling of excreta leads to its partial loss in distal sections due to the peristalsis of duodenum. However, it is of note that passive positioning of the tube, achieved by the weight of the olive in a distal part of the tube and peristalsis of gastrointestinal tract, leads to excessive time required for positioning of the tube and the need to perform radiographic correction of its position, wherein it is difficult to provide an appropriate positioning of the tube's channels for sampling pancreatic juice and bile. The use of stimulating drugs, such as, for example, secretin, cannot be regarded as an adequate solution since they only increase the bicarbonate buffer excretion provided by cells of the pancreas, i.e., only the activity of the epithelial cells covering the ducts is stimulated, and most of the exocrine gland apparatus remains inactive. Therefore, the Dreiling probe does not allow for stimulation of the function of adenocarcinomas, mucinous neoplasms and neuroendocrine tumors. Furthermore, the Dreiling tube is an alternative to the endoscopic retrograde cholangiopancreatography and may be used only to diagnose chronic pancreatitis, wherein intraductal mucinous and cystous formations and carcinomas cannot be revealed in situ by using the Dreiling tube. Furthermore, when the Dreiling tube is used, it is quite difficult to evaluate the choleresis due to the passage of a part of bile or pancreatic juice through the duodenum beside the Dreiling tube.

Furthermore, known in the art is a nasopancreatic stent inserted into the main duct of pancreas by using an endoscopic technique (Osnes M., Petersen H., Schrumpf E. Comparison of juice obtained during duodenal aspiration and cannulation of the main pancreatic duct after stimulation with exogenous secretin in man. Scand J Gastroenterol 1978; 13 (4): 453-458; Minami T., Hanada K., Hirano N. et al. Clinical Usefulness of Serial Pancreatic-Juice Aspiration Cytological Examination and Endoscopic Ultrasound-Guided Fine-Needle Aspiration in Small Pancreatic Cancer. Gastroenterology 2017; 152 (5, Supplement 1): S897; Bi Y., Ji B., Raimondo M. How to suction pancreatic juice from the duodenum: Endoscope, catheter, or cap-assisted. Gastrointestinal Endoscopy 2017; 86 (6): 1157-1159). During an endoscopic papilosphincterotomy, the nasopancreatic stent allows a probe to be inserted and, therefore, allows for pancreatic secretion to be obtained. The disadvantage of the nasopancreatic stent is its traumatic nature, leading to the development of pancreatitis, cholangitis or obstructive jaundice. In 3-10% of cases, an endoscopic retrograde cholangiopancreatography may cause acute pancreatitis, so that in this case all patients have to go through a special preventive therapy. Furthermore, during the papilosphincterotomy, a large vessel may be damaged, thereby causing bleeding. It is to note that the nasopancreatic stent may be used only in highly specialized centers by a specialist experienced with the procedure.

Furthermore, known in the art is a dual-lumen duodenal probe inserted through a nasal passage under control of an endoscope, allowing for the duodenal probe to be advanced through a pyloroduodenal area (Bi Y., Ji B., Raimondo M. How to suction pancreatic juice from the duodenum: Endoscope, catheter, or cap-assisted? Gastrointestinal Endoscopy 2017; 86 (6): 1157-1159; Go V. L., Hofmann A. F., Summerskill W. H. Simultaneous measurements of total pancreatic, biliary, and gastric outputs in man using a perfusion technique. Gastroenterology 1970; 58:321-328). A disadvantage of the duodenal probe is the impossibility of selective sampling of pancreatic juice and bile, and lack of a barrier for mixing of the excreta with intestinal and gastric contents, which leads to activation of pancreatic enzymes and digestion of biological material in the probe. Furthermore, it is of note that design of the duodenal probe does not allow one to influence the outflow of pancreatic juice from the pancreas, so that in order to obtain the juice and perform its examination, pancreas has to be stimulated by drugs such as secretin.

Furthermore, known in the art is a pancreatic juice aspiration device configured to aspirate pancreatic juice by using an endoscope formed as a cap fitted on a fibroscope, wherein the cap allows for the pancreatic secretion to be collected after stimulation of the organ's exocrine function (Suenaga M., Sadakari Y., Almario J. A. et al. Using an endoscopic distal cap to collect pancreatic fluid from the ampulla (with video). Gastrointest Endosc 2017; 86: 1152-1156). Disadvantages of the aspiration device are as follows: the complexity of the procedure since it can be implemented only in highly specialized centers, and a small amount of the collected pancreatic secretion (wherein the available amount of pancreatic secretion is a critical aspect of a molecular test). In this case, the procedure based on the use of the aspiration device is performed by using anesthetic aid, wherein the procedure cannot be performed for a long time.

Known in the art is a method of diagnosing viral hepatitis in blood by using serological and molecular genetic methods. Nevertheless, in 20% of cases, a disease remains unverified since it is conditioned by a life cycle of a virus and by its tropism both to hepatocytes and to the epithelium of the bile ducts, causing viral cholangitis and the disease chronization (Shakhgildyan I. V., Mikhailov M. I., Onishchenko G. G. Parenteral viral hepatitis (epidemiology, diagnosis, prevention). GOU VUNMTS MZ RF, Moscow, Russia, 2003; Burgart L. J. Cholangitis in Viral Disease. Mayo Clinic Proceedings 1998; 73 (5): 479-482). The diagnostic method does not allow for obtaining a bile for further laboratory analysis.

Known in the art is a method of diagnosing diseases of the liver (viral hepatitis, autoimmune hepatitis, sclerosing cholangitis, liver tumors) by performing a percutaneous biopsy followed by histological and molecular genetic analysis (Bunt E. M. Liver Biopsy Diagnosis of Hepatitis: Clues to Clinically-Meaningful Reporting. Mo Med 2010; 107 (2): 113-118). In some cases, such a method leads to complications (bleeding, biliary peritonitis). At the same time, it does not allow one to reveal pathologic changes in about 30% of cases. Furthermore, such a method is used exclusively for primary diagnostics, and practically never used repeatedly, for example for monitoring the dynamics or development and progression of a disease.

Furthermore, known in the art is a method of treating acute pancreatitis by stenting the main pancreatic duct to restore the efflux of enzymes of the pancreas. Such a method is used for treating and preventing pancreatitis after performing the endoscopic retrograde cholangiopancreatography (Mozharovsky V. V., Mutnykh A. G., Zhukov I. N., Mozharovsky K. V. Stenting of the main pancreatic duct influences the treatment results obtained for patients with an acute pancreatitis. Pirogov Russian Journal of Surgery. 2019; (9): 13-17; Dumonceau J. M., Andriulli A., Elmunzer B. J. et al. Prophylaxis of post-ERCP pancreatitis: European Society of Gastrointestinal Endoscopy (ESGE) Guideline—updated June 2014. Endoscopy 2014; 46 (9): 799-815). A disadvantage of the method is in that stenting procedure can be performed only in highly specialized centers, wherein in some cases the procedure cannot be performed at all due to anatomical features of patient's duct system. Furthermore, the procedure does not solve the problem of pancreatitis due to the disruption of the outflow of pancreatic juice associated with paresis of duodenum.

Furthermore, intestinal fistulas occur on average in 1-3% of patients after performing operations on abdominal cavity organs (Smotrin I. S. Obturating agents for treating gastrointestinal fistulas. Journal of the State Medical University for Practicing Physicians 2007; 4). The overall mortality associated with this pathology ranges from 16.5% to 57.5%, and the postoperative mortality associated with this pathology ranges between 10% and 21.4%, wherein the greatest mortality is observed for disembodied intestinal fistulas and ranges between 36% and 71.7%. The mortality barely reaches 4% for embodied intestinal fistulas.

Known in the art is a method of treating gastrointestinal fistulas by using systems for producing a negative pressure, wherein the method is based on the constant evacuation of all pathologic discharge from abdominal cavity, thereby healing defects (Bobkiewicz A., Walczak D., Smoliński S. et al. Management of enteroatmospheric fistula with negative pressure wound therapy in open abdomen treatment: a multicenter observational study. Int Wound J 2017; 14 (1): 255-264; D'Hondt M., Devriendt D., Van Rooy F. et al. Treatment of small-bowel fistulae in the open abdomen with topical negative-pressure therapy. Am J Surg 2011; 202 (2): e20-24). Furthermore, in case when the method is used, statistics collected by the same authors indicate that such defects are not closed (not healed) in 30-47% of cases due to contents constantly produced and received from a lumen of a hollow organ.

Furthermore, there are a large number of different obturator-like devices aimed at disconnecting the lumen of a hollow organ with a defect in a wall. However great variety of such devices suggests the difficulty of their implementation and achieving desired effects. Furthermore, all such well-known devices are aimed only at disconnecting the lumen of a hollow organ with a defect in a wall and do not aim to impact an area adjacent to the wall defect (Vitsyn B. A., Blagitko E. M. Formed and unformed external intestinal fistulas. Nauka, Novosibirsk, Russia. 1983; 142 p.; Makarenko T. P., Bogdanov A. V. Gastrointestinal fistulas. Medicine, Moscow, Russia. 1986. 144 pp.; USSR AS 764685, M. class A 61 M 27/00. Obturator for temporary closure of a gastrointestinal fistula/VM Udod and E. G. Karsten. By application 2723729/28-13. Declared Dec. 22, 1978. Publ. Sep. 23, 1980. BI 35).

Known in the art are methods of treating gastrointestinal hemorrhages that are based on the usage of endoscopic methods, clipping, ligation, injection of sclerosants or vasoactive drugs into the mucosal layer, and also coagulation using thermal and electrical methods (Anjiki H., Kamisawa T., Sanaka M., Ishii T., Kuyama Y. Endoscopic hemostasis techniques for upper gastrointestinal hemorrhage: A review. World J Gastrointest Endosc 2010; 2 (2): 54-60). Furthermore, in case of bleeding from a putrescent tumor, necrotizing esophagitis or nonspecific erosive colitis, i.e., in case when the diffuse mucosa bleeding occurs without an obvious source, such known methods remain ineffective, while they also do not allow for objective control of stability of hemostasis.

Known in the art is a method of diagnosing aneurismal dilatations of vessel by administering intraluminal endovascular catheters with or without stents, involving guiding the catheter under control of a fluoroscopy and obturating an aneurism with a stent (Roszelle B. N., Nair P., Gonzalez L. F., Haithem Babiker M., Ryan J., Frakes D. Comparison among different high porosity stent configurations: hemodynamic effects of treatment in a large cerebral aneurysm. J Biomech Eng 2014; 136 (2): 021013). However, if this known method is implemented, during the positioning of a catheter in a correct manner and identifying a defect, a circumferential blood flow may suffer, and hemorrhage from an aneurysm cavity may continue. Therefore, existing analogues differ from the claimed technique.

Known in the art is a method for detecting injuries of hollow organs by administering various coloring or radiopaque substance into a hollow organ (Ozimok C. J., Mellnick V. M., Patlas M. N. An international survey to assess use of oral and rectal contrast in CT protocols for penetrating torso trauma. Emerg Radiol 2019; 26 (2): 117-121; Broder J. S., Hamedani A. G., Liu S. W., Emerman C. L. Emergency department contrast practices for abdominal/pelvic computed tomography-a national survey and comparison with the American college of radiology appropriateness criteria. J Emerg Med 2013; 44 (2): 423-433). However, the manipulation itself only allows an injury to be ascertained, wherein this is not always possible since the success of ascertaining an injury strongly depends on the location of an injury and specifics of administration of a substance. In other words, this known method does not always allow for an injury of a hollow organ to be accurately located and does not allow for treatment of this injury.

### SUMMARY OF INVENTION

An objective of the present disclosure is to develop a catheter and a method for isolating a region in a hollow organ of a mammal, the catheter solving at least the above technical problems.

To achieve the objective of the disclosure, as embodied and broadly described herein, in one aspect of the present disclosure, a catheter for isolating a region in a mammal hollow organ is disclosed. The catheter can include an elongate body having a proximal end, a distal end, and a central lumen disposed within the elongate body. The elongate body can define a longitudinal axis, and the entire elongate body may be configured to be inserted into a lumen of a mammal hollow organ. The proximal end of the elongate body can define a first opening of the elongate body and the distal end can define a second opening of the elongate body. The catheter can include a plurality of proximal extensions coupled to the proximal end of the elongate body at a tapered section of the proximal end. The tapered section can be tapered with respect to the elongate body, and the plurality of proximal extensions can include a plurality of individual channels that are continuous with the elongate body such that the plurality of individual channels are embedded within the elongate body. The catheter can include two balloons that are individually and separately disposed along the elongate body. The two balloons can be configured to be inflated to isolate an interior of the hollow organ therebetween. One of the plurality of individual channels can include a functional channel that includes one or more functional openings provided in the elongate body between the two balloons. The functional channel can be designed to allow a negative pressure to be produced in the isolated interior to take a fluid or gaseous medium therefrom. The functional channel can be designed to allow a liquid or gaseous medium to be supplied into the isolated interior. The plurality of channels can also include a second channel that can extend in the elongate body to deliver a fluid or gaseous medium to the balloons to provide for their inflation. According to some embodiments, the second channel may provide a fluid or gaseous medium to both of the balloons, while in other embodiments, a separate channel may be provided for delivering fluid or gaseous medium to each balloon separately. A technical effect of providing a single channel to inflate both balloons is that the fluid or gaseous medium can be uniformly redistributed during the passage of a peristaltic wave of a hollow organ, such as the intestine or esophagus. A technical effect of providing a separate channel for each balloon is that each balloon may be inflated and/or deflated independently of one another, which may provide a technical benefit for certain vascular applications. In particular, such a separate inflation or deflation of balloons may be beneficial for better positioning of the catheter in the hollow organ. Also, full or partial deflation of one of the balloons may be beneficial in order to check the quality of surgical procedure performed on a wall of a hollow organ such as blood vessel (e.g., to determine the leakage through the wall of a hollow organ and assess the quality of stitching of the wall). Also, complete or partial deflation of one of the balloons may be beneficial if a branching of blood vessels occurs inside the isolated area of a larger vessel to allow for perfusion of blood into the branching vessel.

The functional channel in the catheter body provides further technical effect, by providing sanitization of the hollow organ of the mammal (in particular, sanitization of the esophagus, stomach, duodenum, small intestine, large intestine, as well as of air in respiratory tracts, urine in urinary tracts or blood in vessels), while simultaneously enabling the provision of medical products to the isolated region of the hollow mammal organ. An enteral nutrition mixture, which may provide the organism with basic nutrients, energy, vitamins, macronutrients, micronutrients and/or etc., can also be provided to the interior of the hollow organ while sampling a biological fluid or biological gaseous medium from the isolated region of the hollow organ or supplying a liquid or gaseous medium to the isolated region of the hollow organ. These features allow the catheter to be inserted into the lumen of the hollow organ of the mammal for a long time. According to some embodiments, the enteral nutrition mixture may be supplied directly to the isolated area, while in other embodiments, the enteral nutrition may be supplied to the hollow organ but outside of the isolated area, for example by supplying the enteral nutrition mixture through the first opening of the elongate body.

Delivery of a fluid or a gaseous medium into catheter balloons through the second channel provided in the catheter body to provide inflation thereof provides a technical effect, which consists of a simplification of maintaining the sizes of balloons required to isolate the interior in the lumen of a hollow organ where the catheter is inserted and simplification of the regulation of sizes or degree of inflation of catheter balloons.

A technical effect provided by the catheter for isolating a region in a hollow organ of a mammal according to the present disclosure is prevention or exclusion of formation of congestive and/or inflammatory processes in the hollow organ due to the accumulation of mucus and other biological contents being specific for the hollow organ outside the isolated interior defined by the balloons. In particular, in the present disclosure, formation of congestive and/or inflammatory processes in the hollow organ is prevented or excluded due to the fact that mucus and other biological contents being specific for the hollow organ, when accumulated in the hollow organ, may enter the first opening and/or the second opening provided in the catheter body outside the isolated interior defined by the balloons, and may leave through the second opening and/or first opening provided in the catheter body outside the isolated interior defined by the balloons.

In some embodiments, the first opening can include an oblique opening within the elongate body of an angle between approximately 15 degrees to approximately 65 degrees with respect to the longitudinal axis defined by the elongate body. A technical effect provided by the oblique opening of the first opening can be an increased surface area of the first opening which facilitates the unrestricted flow or perfusion of biological fluids through the elongate body of the catheter, thereby increasing connectivity between areas proximal and distal to the isolating balloons. A further technical effect provided by the oblique opening of the first opening can be the facilitation of atraumatic removal of the catheter as compared to the removal of a catheter lacking the oblique first opening.

In some embodiments, the second opening can include an oblique opening within the elongate body of an angle between approximately 15 degrees to approximately 65 degrees with respect to the longitudinal axis defined by the elongate body. A technical effect provided by the oblique opening of the second opening can be an increased surface area of the second opening which facilitates the unrestricted flow or perfusion of biological fluids through the elongate body of the catheter, thereby increasing connectivity between areas proximal and distal to the isolating balloons. A further technical effect provided by the oblique opening of the second opening can be facilitation of atraumatic insertion of the catheter as compared to the insertion of a catheter lacking the oblique second opening.

In some embodiments, the central lumen can be configured to facilitate bidirectional flow through the first opening and the second opening.

In some embodiments, the second opening can further include an opening at the distal end configured to facilitate insertion of the elongate body into the lumen of the mammal hollow organ.

In some embodiments, each of the plurality of channels are hermetically isolated from each other and from the central lumen of the elongate body. The hermetically isolated channels in the catheter provide a further technical effect by preventing contents of the functional channel from being contaminated by contents from other catheter channels which would negatively influence the reliability or representativity of laboratory-instrumental analysis results obtained for the biological fluid or biological gaseous medium of the isolated region. The hermetically isolated channels also provide a further technical effect by preventing contents from the other channels from mixing with a liquid or gaseous medium supplied to the isolated region of the mammal hollow organ.

In some embodiments, the catheter can include two enclosing projections on the elongate body. The one or more functional openings can be positioned between the two enclosing projections. In some embodiments, the enclosing projections can be ring-shaped and positioned adjacent to or against the one or more functional openings.

In some embodiments, the projections enclosing the functional opening provided in the catheter body provide a further technical effect by preventing suction of a hollow organ tissue, in particular a mucosal tissue of a hollow organ, into the functional opening since the enclosing projections do not allow the functional opening to contact directly with the tissue or approach the tissue for a distance appropriate for suction thereof when a negative pressure is produced in the hollow organ interior isolated by the catheter balloons. In some embodiments, the shape of the enclosing projections and their positions in relation to the functional opening further contribute to the technical effect of the prevention of suction of hollow organ tissue into the functional opening and, therefore, preventing the blockage or occlusion of the functional opening.

In some embodiments, the elongate body can include a net member that is designed that it at least partly encloses the catheter part defined by the balloons and covers the one or more functional openings. In some embodiments, the net member can be attached to the balloons such that the net member can become strained when the balloons are inflated. In some embodiments, the net member can include a compression member, the compression member integrated into the net member and configured to provide a compressive force on the balloons when the compression member is pulled proximally, thereby facilitating deflation of the balloons. According to some embodiments, the compression member may be attached to the net member at a point just distal of the proximal inflatable balloon, and the net member may be compressed when the compression member is pulled in a proximal direction, thereby stretching the material of the net member against the elongate body and facilitating the deflation of the inflatable balloons. In other embodiments, the compression member may be integrated into the material of the net member in a helical configuration (e.g., wrapping around the circumference of the net member) such that when the compression member is pulled proximally it provides a force that constricts the circumference of the net member, thereby facilitating deflation of the inflatable balloons. In some embodiments, the net member may be unattached to the elongate body of the catheter, and may be held in place due to having a circumference that is less than a circumference of the catheter body or the circumference of the inflatable balloons. In some embodiments, the net member may be attached to the elongate body of the catheter by either one or both of the inflatable balloons. In some embodiments, the net member may be attached to one or more protrusions disposed on the outside of the elongate body of the catheter.

A further technical effect provided by the catheter for isolating a region in a hollow organ of a mammal according to the present disclosure is prevented suction of a tissue of the hollow organ and/or the net member into the functional opening of the catheter body and, therefore, prevented blockage or occlusion of the functional opening. In some embodiments of the present disclosure, suction of the hollow organ tissue and/or the net member into the functional opening is prevented due to the fact that the net member in the strained state covers the functional opening and is positioned in a distance from the functional opening. In other embodiments of the present disclosure, suction of the hollow organ tissue into the functional opening is prevented due to the two enclosing projections on the elongate body that are configured to space the functional opening from the tissue wall of the hollow mammal organ. A further technical effect provided by the catheter according to certain embodiments of the present disclosure is facilitating the deflation of the balloons by using a compression member integrated into the net member. The compression member may be used to further deflate the balloons prior to the removal of the catheter from a patient.

In some embodiments, the tapered section can include a reduction in diameter of between approximately 20 percent and approximately 90 percent with respect to a diameter of the elongate body at a point where the elongate body ends and the plurality of proximal extensions extend out from the elongate body of the catheter. A technical effect provided by the proximal extensions includes increased long-term comfort and wearability for a patient using a catheter according to the present disclosure.

In another aspect, a system for isolating a region in a hollow organ of a mammal is disclosed. The system can include the catheter for isolating the region in the hollow organ of the mammal according to any of the above-described embodiments and a functional device connected to the functional channel to allow the air to be evacuating therefrom and/or a liquid or gaseous medium to be supplied thereto.

In another aspect of the present disclosure, a method of isolating a region in a hollow organ of a mammal is disclosed. The method can include inserting catheter according to any of the above-described embodiments into a lumen of the hollow organ of the mammal, inflating the balloons to isolated an interior of the mammal hollow organ therebetween, and delivering through the functional channel and the one or more functional openings of the catheter a negative pressure in the isolated interior for taking therefrom a biological fluid or a biological gaseous medium specific to the hollow organ or specific of an organ connected to the hollow organ, or supplying, by means of the functional channel and the one or more functional openings, a liquid or gaseous medium to the isolated interior. According to some embodiments, the method may include controlling the catheter insertion by an endoscope or radiographic equipment.

According to some embodiments, the catheter insertion control provided by the endoscope or the radiographic equipment provides a further technical effect which is greater accuracy of catheter placement in the lumen of a hollow organ of a mammal, enabled visual control of an inflation degree of the catheter balloons and prevented damage of a hollow organ tissue, in particular a mucosal tissue of a hollow organ, when the catheter is advanced to a required position in the lumen of a hollow organ.

The catheter for isolating the region in the hollow organ of the mammal according to any one of the above-described embodiments of the present disclosure may be used for diagnosing or monitoring the development or monitoring the treatment of at least one disease selected from a group comprising: bleeding, haemostasis, gastritis, acute and chronic pancreatitis, autoimmune pancreatitis pancreatic neoplasms and cancer, bile duct cancer, cholangiocarcinoma, hepatocellular cancer, cholangitis, cholelithiasis, defects of a hollow organ wall, fistula of hollow organs, autoimmune hepatitis, infectious hepatitis, sclerotic cholangitis, Crohn disease, ulcerative colitis, eosinophilic esophagitis, aneurismal or diverticular protrusions of a hollow organ wall, bleeding of hollow organs, bleeding into hollow organs, strictures of hollow organs, esophagus neuromuscular disorders, stomach neuromuscular disorders, duodenum neuromuscular disorders, neuromuscular disorders of the small intestine and colon and rectum, neuromuscular disorders of bile ducts, neuromuscular disorders of urinoexcretory ways, and neuromuscular disorders of respiratory tracts, vascular thrombosis and embolization, vessels' malformations.

Furthermore, the catheter for isolating the region in the hollow organ of the mammal according to any one of the above-described embodiments of the present disclosure may be used for treating at least one disease selected from a group comprising: bleeding, pancreatitis, cholangitis, gastrointestinal wall defects, fistulas of hollow organs, vascular defects, vascular aneurisms, vascular thrombosis and embolization, defects of a hollow organ wall, bleeding of hollow organs, bleeding into hollow organs, gastrointestinal hemorrhages, bleeding from the windpipe (trachea), bronchial bleeding, pulmonary hemorrhage, uterine bleeding, vessels' malformations, Crohn disease, ulcerative colitis, eosinophilic esophagitis, malignant diseases that occur in hollow organs and organs that are connected to hollow organs.

Furthermore, the catheter for isolating the region in the hollow organ of the mammal according to any one of the above-described embodiments of the present disclosure may be used to isolate a hollow organ area when performing a surgical procedure. According to some embodiments, the hollow organ area to be isolated when performing a surgical procedure may be a blood vessel.

### BRIEF DESCRIPTION OF THE DRAWINGS

While the specification concludes with claims particularly pointing out and distinctly claiming the present disclosure, it is believed the same will be better understood from the following description taken in conjunction with the accompanying drawings, which illustrate, in a non-limiting fashion, the best mode presently contemplated for carrying out the present disclosure, and in which like reference numerals designate like parts throughout the drawings, wherein:

FIG. 1A schematically illustrates an exemplary stent-like catheter for isolating a region in a hollow organ of a mammal according to aspects of the present disclosure;

FIG. 1B illustrates an exemplary stent-like catheter for isolating a region in a hollow organ of a mammal including a plurality of peripheral openings in the catheter body, according to aspects of the present disclosure;

FIG. 1C illustrates an exemplary full-body catheter for isolating a region in a hollow organ of a mammal, according to aspects of the present disclosure;

FIG. 2 illustrates a functional opening provided in a part of the catheter of FIG. 1A 1B, 1C, and FIG. 8;

FIG. 3A illustrates a catheter with an oblique second (e.g., distal) opening, according to aspects of the present disclosure;

FIG. 3B illustrates a catheter with a stub-shaped second (e.g., distal) opening, according to aspects of the present disclosure;

FIGS. 4A and 4B illustrate a catheter with an oblique first (e.g., proximal) opening, according to aspects of the present disclosure;

FIG. 5A illustrates a catheter with a net member when the isolating balloons are in a deflated configuration, according to aspects of the present disclosure;

FIG. 5B illustrates a catheter with a net member when the isolating balloons are in an inflated configuration, according to aspects of the present disclosure;

FIG. 6A illustrates a catheter with a net member and a compressive member integrated into the net member when the isolating balloons are in an inflated configuration, according to aspects of the present disclosure;

FIG. 6B illustrates a catheter with a net member and a compressive member integrated into the net member when the isolating balloons are in a deflated configuration, according to aspects of the present disclosure;

FIG. 7A illustrates a catheter with a net member and a simplified compressive member when the isolating balloons are in an inflated configuration, according to aspects of the present disclosure;

FIG. 7B illustrates a catheter with a net member and a simplified compressive member when the isolating balloons are in a deflated configuration, according to aspects of the present disclosure; and

FIG. 8 illustrates another exemplary stent-like catheter for isolating a region in a hollow organ of a mammal with a shortened elongate body, according to aspects of the present disclosure.

### DETAILED DESCRIPTION

In the context of this document, unless explicitly stated otherwise, the term “patient” means first of all a potentially sick person (a member of the mammalian class) seeking medical advice or remaining under medical observation to have a disease diagnosed and/or treated, wherein the term “patient” also means potentially sick mammalian animals remaining under medical observation to diagnose and/or treat their disease.

Furthermore, in the context of this document, unless expressly stated otherwise, the term “mammal” means a human or an animal, in particular anthropoid and non-human primates, dogs, cats, horses, camels, donkeys, cows, sheep, pigs, and other well-known mammals.

Furthermore, in the context of this document, unless expressly stated otherwise, the term “user” means any suitably skilled health care professional authorized to insert the catheter according to the present disclosure into a hollow organ of a mammal (in particular, a human hollow organ), remove the catheter according to the present disclosure from a hollow organ of a mammal and/or manipulate the catheter according to the present disclosure inserted into a hollow organ of a mammal, wherein the healthcare professional may be, for example, surgeon, oncologist, endoscopist, thoracic surgeon, angiosurgeon, urologist, veterinarian, etc.

Currently, one of the most advanced methods of diagnosing and treating inflammatory, autoimmune, and malignant diseases that occur in hollow organs and organs that are connected to hollow organs, more particularly diseases of pancreas, bile ducts, gallbladder, liver, gastrointestinal tract, and also defects and injuries of mammal hollow organs such as fistulas, strictures, aneurismal and diverticular dilatations, etc., is a liquid biopsy based of biological liquids and fluids sampled from a specific hollow organ of a mammal. In particular, a liquid biopsy can be used to diagnose and/or treat pancreatic cancer of a mammal at an early stage by detecting pathologic cells, tumor DNA, RNA, proteins, peptides, metabolites, and/or circulating tumor exosomes in the biological liquid being sampled (such as blood and pancreatic juice).

In order to efficiently perform a liquid biopsy, it is necessary to have a sufficient quantity of sampled material to perform the diagnosis. Another important factor for performing the liquid biopsy is the localization of biological samples to the hollow organ of interest. If a biological sample is taken without effective isolation of the hollow organ being sampled, the diagnostic biological material isolated from a collected sample may be typical of diseases and/or cancers of different organs, thereby obfuscating the results of the targeted biopsy.

In view of the above, to diagnose a cancer, mucinous and intraepithelial neoplasms of the pancreas, pancreatic juice is the most appropriate diagnostic biological liquid to sample to detect circulating tumor cells, DNA, RNA, proteins, peptides, metabolites, exosomes, etc. therein. Isolating a duodenum around a major and/or minor duodenal papilla to sample pancreatic juice is a difficult problem which is effectively solved by catheter 100 and/or catheter 200 according to any one of the below-described embodiments of the present disclosure. Structural and design features and functionalities of catheter 100 and/or catheter 200 according to the present disclosure are described in detail below in context of solving the illustrative task of collecting pancreatic juice from duodenal papillae; however, the scope of the present invention is not limited to collecting pancreatic juice and treatment/diagnosis of the pancreas. Therefore, it is clear for one skilled in the art that the catheter 100 and/or catheter 200 according to any of the above-described embodiments can be similarly inserted into a lumen of any another hollow (tubular) internal organ of a patient, in particular mammals, for example in the esophagus, stomach, duodenum, small intestine, large intestine, respiratory tracts, urinary tracts (urogenital system tracts), veins, arteries, vagina, uterus, uterine (Fallopian) tubes, vertebral canal or any appropriate internal tubular organ of a patient, the patient tubular organ being related to a corresponding functional system (apparatus of organs) of a mammal organism from a group of systems including: digestive system, respiratory system, urinary and reproductive systems (combined into the genitourinary system or urogenital system), endocrine system, circulatory system and immune system, and skeletal system.

It is to note that the minor duodenal papilla and the major duodenal papilla (also referred to in the art as Santorini's papilla and Fateri's papilla, respectively) each represent an anatomical structure in the form of a hemispherical, conical or flattened elevation located at the end of the longitudinal fold of the mucous membrane in the middle of the descending part of the duodenum, in particular about 12-14 cm below the pylorus, wherein in most cases one opening common for the bile and pancreatic ducts is exposed to the duodenum lumen, and in other cases the pancreatic duct is exposed 2-4 cm above the duodenal papilla. A hepato-pancreatic ampoule is located in the duodenal papilla, the ampoule being used for receiving bile and digestive juices of the pancreas and contains the Oddi sphincter regulating the flow of bile or pancreatic juice into the duodenum and preventing the intestinal contents from entering the bile and pancreatic ducts. Therefore, the major duodenal papilla in the duodenum of a mammal is usually 12-14 cm below the pylorus, and the minor duodenal papilla is 2-4 cm above the major duodenal papilla.

In particular, in order to provide the flow of pancreatic juice into the lumen of duodenum it is necessary to create physiological negative pressure around the Fateri's papilla or major duodenal papilla at a level of 40-100 mm wg, which is normally achieved by peristalsis of duodenum (Teesalu S. Physiology of digestion. Tartu. Tartu State University. 1987; p. 84; The Pancreas. Third edition. Blackwell, UK. 2018; 1300 p.). Another criterion is the need for isolated sampling of a pancreatic juice with inactive digestive enzymes, without gastric and duodenal juice and contents, since the presence of gastric and duodenal juice and contents results in activation of enzymes and digestion of cells, DNA, RNA, proteins, peptides, metabolites, and exosomes necessary for diagnosis and treatment of diseases of pancreas.

The below figures schematically illustrate a catheter 100 and/or catheter 200 for isolating a region in a hollow organ according to aspects of the present disclosure, wherein catheter 100 and/or catheter 200 are catheters to be inserted by a user into a lumen of a hollow organ, and wherein a housing or a body of the catheter 100 is formed as a flexible hollow stent-like tube having dimensions, in particular a length and a thickness, suitable for user-assisted insertion or advancement thereof within the lumen of a hollow organ towards a placement site and catheter 200 is a full-bodied version of the catheter device. A user manipulating either catheter 100 and/or catheter 200 may be an appropriately skilled healthcare professional, such as, for example, surgeon, oncologist, endoscopist, thoracic surgeon, angiosurgeon, urologist, veterinarian, etc. The catheter 100 and/or catheter 200 for isolating the region in the hollow organ may be used for any patient, in particular any human or animal.

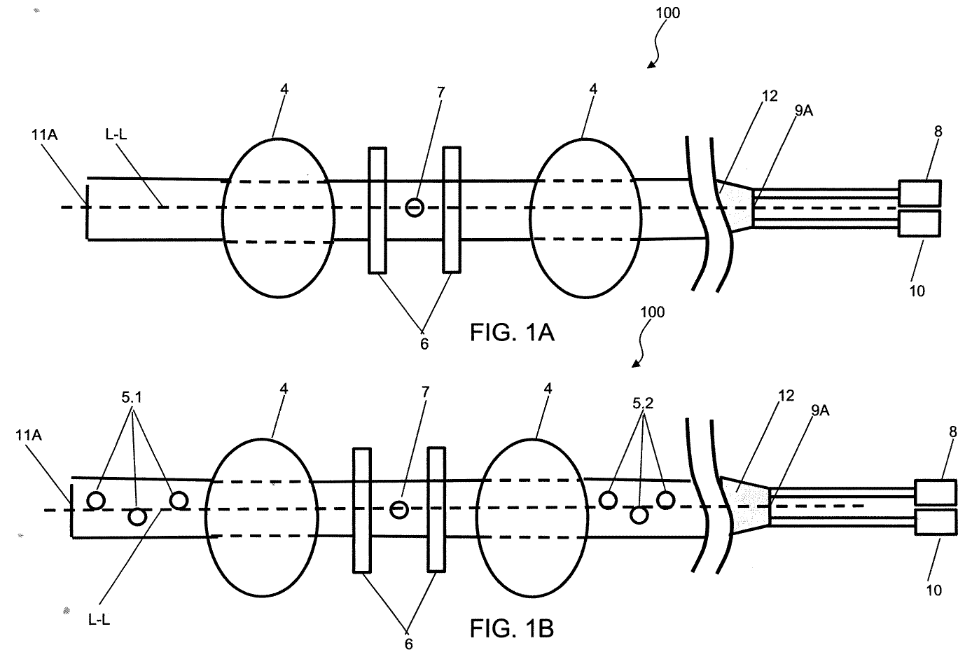

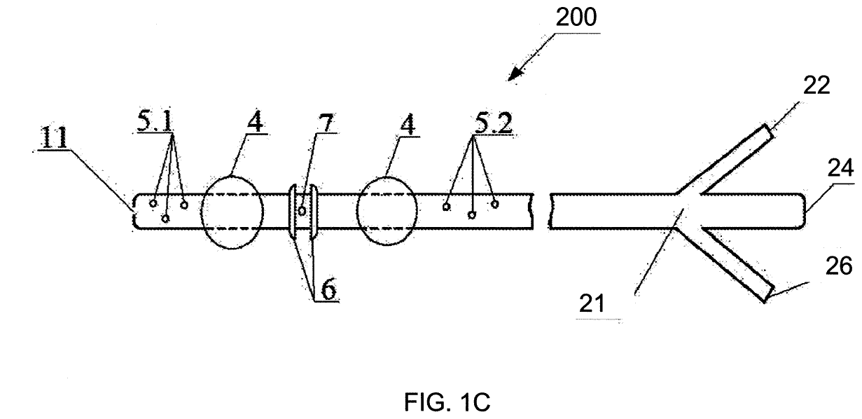

FIG. 1A illustrates catheter 100 for isolating a region in a hollow organ according to aspects of the present disclosure. More particularly, FIG. 1A shows catheter 100 having an elongate body defining a longitudinal axis L-L, a plurality of proximal extensions 8, 10, a first opening 9A on the proximal end of the elongate body, a tapered section 12 of the elongate body, which may be beneficial for inserting and/or removing the device from a mammal hollow organ, isolating balloons 4, a second opening 11A on the distal end of the elongate body, a functional opening 7, and two projections 6 on either side of functional opening 7. The second opening 11A (e.g., the distal end) can be configured for insertion into a patient followed by advancing catheter 100 to a placement site within the lumen of a patient's hollow organ, in particular, in the gastrointestinal tract, bile ducts, respiratory tracts, urinary system, patient vessels, a cavity related to uterine and vagina, etc. The entire elongate body of catheter 100 can be configured to be inserted into the patent, while the proximal extensions 8, 10, can be configured to remain outside of the patient's body. In particular, when catheter 100 is used for isolating a region in the patient's duodenum, catheter 100 can be inserted by its distal end (second opening 11A) into the patient's nasal passage followed by advancing catheter 100 along a patient's duodenum to a placement site in the patient's duodenum.

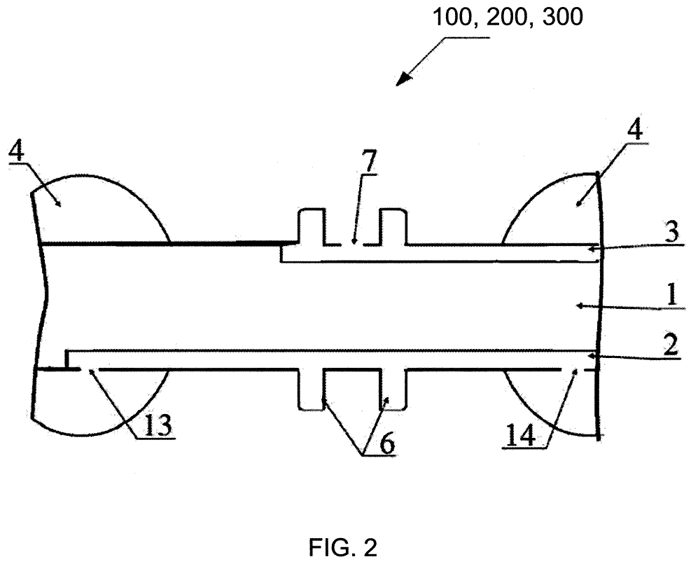

As shown in FIG. 1A, catheter 100 can include three distinct openings: first (central) opening 9A, and two peripheral openings provided as proximal extensions 8 and 10. It should be noted, that according to some embodiments consistent with the present disclosure there may be any number of proximal extensions configured to perform various functions, and catheter 100 is not limited to just two proximal extensions 8 and 10. Each of opening 9A and proximal extension 8 and 10 can include a corresponding channel (as shown in FIG. 2, infra) where each of proximal extension 8, 10, and opening 9A are hermetically isolated from each other. The free ends of openings 8, 9A, and 10 can be configured to connect an appropriate functional appliance during operation of catheter 100.

First opening 9A can be configured to be positioned within a hollow organ and can facilitate bidirectional flow of fluids and gases through the elongate body of catheter 100. That is, fluids and gases can pass from the proximal first opening 9A distally through second opening 11A and vice versa from the distal end through central opening 9A. The structure of catheter 100 allows for the central channel corresponding to first opening 9A and second opening 11A to have a maximally enlarged diameter to allow for the unrestricted flow of fluids and/or gasses through the elongate body of catheter 100.

According to some embodiments, the first opening 9A may be connected to a proximal extension (not shown) that is configured to connect or join an appropriate (first) functional appliance or device thereto, wherein the (first) functional device may be any device known in the art for supplying a gaseous medium or fluid (not shown), for example an enteral nutrition mixture. The device for supplying a gaseous medium or fluid as connected to the central terminal 9A may be, for example, an injection syringe filled, for example, with an enteral nutrition mixture to provide patient's organism with basic nutrients, energy, vitamins, macronutrients, micronutrients and/or etc., or a medical dropper filled, for example, with an enteral nutrition mixture, or other devices and appliances, including automated or semi-automated, suitable to connect the central terminal 9A thereto and supply a gaseous medium or fluid, for example an enteral nutrition mixture, to the central terminal 9A. Therefore, the device for supplying a gaseous medium or fluid connected or attached to the central terminal 9A may be used to provide, for example, nutrition support or clinical nutrition to a patient when the catheter 100 is inserted within the lumen of duodenum (or other part of gastrointestinal tract). Furthermore, the device for supplying a liquid or gaseous medium, when connected or attached to the central terminal 9A, may be used, for example, to sanitize the stomach and patient's duodenum.

Proximal extension 8 can be designed to connect or attach to an appropriate (second) functional appliance or device thereto, wherein the second functional device may be implemented, for example, as a medical suction apparatus, an aspiration device or an aspirator (not shown) that includes a storage reservoir or a storage container (not shown) for collecting gaseous medium, liquid and/or fluid, and an air compressor (not shown) for aspirating or evacuating an air or another appropriate gaseous medium. The pressure for aspirating or evacuating the air or another appropriate gaseous medium may be adjusted by a one skilled in the art for corresponding hollow organs on the basis of information disclosed in, for example, in U.S. Pat. No. 6,712,798.

The second functional device that may be attached to proximal extension 8 may be formed as a special device for supplying a gaseous medium or fluid (e.g., medical products), for example an injection syringe filed with a liquid to be supplied, or a medical dropper filled with a liquid to be supplied, or other devices or appliances, which can be automated or semi-automated, suitable for connecting to proximal extension 8 and supplying gaseous mediums or fluids through proximal extension 8.

Proximal extension 10 can be configured to connect or attach to an appropriate (third) functional device, wherein the third functional device can be implemented as a special device for supplying a liquid or gaseous medium under pressure (not shown), for example water or air, in particular an injection syringe filled with a liquid of gaseous medium, or a medical dropper filled with a liquid to be supplied, or other devices and appliances, including either automated or semiautomated devices, that are suitable for connecting to proximal extension 10 and supplying a fluid or gaseous medium through proximal extension 10.

Notably, the first functional device connectable to first opening 9A, second functional device connectable to proximal extension 8, and third functional device connectable to proximal extension 10 of catheter 100 may form a system for isolating a region in a hollow organ of a mammal, which may be used for sampling a fluid or gaseous medium (for example, a biological fluid) being specific of a hollow organ or of an organ connected to a hollow organ of a mammal, and/or may constitute corresponding parts of such a system. In some embodiments, the above system may be used for isolating a region of the duodenum of a mammal for sampling pancreatic juice and/or bile.

Notably, in some embodiments, each of the plurality of proximal extensions (e.g., proximal extensions 8, 10) can be provided as separate proximal extensions that are not connected to any other proximal extension. In other embodiments, each of the plurality of proximal extensions can be bundled together such that each proximal extension (e.g., proximal extensions 8, 10) remains hermetically isolated, but is otherwise bundled or held together in order to facilitate the operation of catheter 100 when catheter 100 is inserted into a hollow organ of a patient.

Catheter 100 can also include two isolating balloons 4 which can be disposed on an outside of the elongate body of catheter 100. Isolating balloons 4 can be formed as expandable or inflatable reservoirs, and the balloons may be spaced at a predetermined distance from each other and from the distal end of catheter 100. Further, catheter 100 can include a functional opening 7 between the isolating balloons 4 in the elongate body of catheter 100. When catheter 100 is inserted into a lumen of a patient's hollow organ, one of the isolating balloons 4, namely the farthest from the proximal end of catheter 100, becomes positioned distally to the area of interest in the hollow organ, and the other isolating balloon 4 is positioned proximal to the area of interest in the hollow organ. The functional opening 7 can be located at the area of interest or next thereto, for example opposite or adjacent to one of the minor and major duodenal papillae between the minor duodenal papilla and the major duodenal papilla, opposite or adjacent to an intestinal fistula, opposite or adjacent to a wall injury, opposite or adjacent to a tumor, opposite or adjacent to a site of inflammation, etc. It should be noted that in some embodiments, functional opening 7 can be provided as a plurality of functional openings 7.

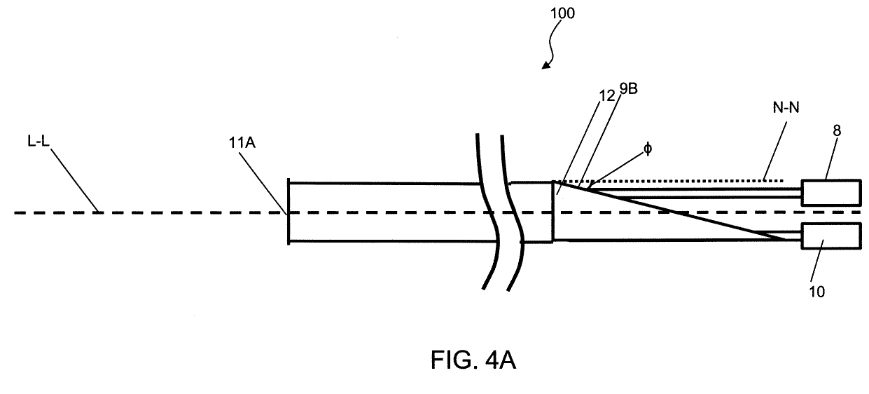

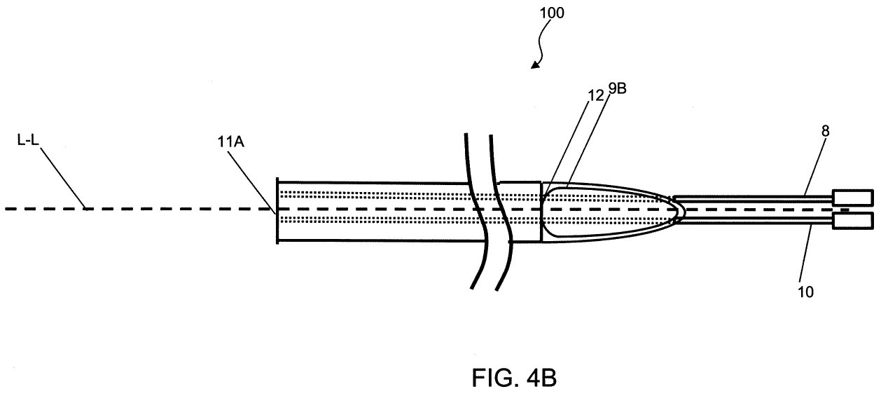

Tapered section 12 of the proximal end of catheter 100 can be configured to reduce the cross-sectional area of catheter 100, thereby facilitating the removal of the elongate body of the catheter from a patient undergoing a procedure involving the utilization of catheter 100. According to some embodiments, first opening 9B may be tapered and/or oblique, as shown in more detail with respect to FIG. 4, to facilitate atraumatic removal of catheter 100 from a patient. In some embodiments, an oblique opening 9B may be configured to increase the cross-sectional area of opening 9B and thereby provide higher rate of flow for fluids and/or liquids passing through the catheter 100. An oblique opening 9B may also facilitate atraumatic removal of catheter 100 from a patient. According to some embodiments, first opening 9A may be a tapered stub shape opening. The slight taper of opening 9A may facilitate atraumatic removal of the catheter from a patient but may not facilitate the higher rate of flow for fluids as does oblique opening 9B.

With respect to FIG. 1B, according to some embodiments consistent with the present disclosure, catheter 100 may be provided with a plurality of auxiliary holes 5.1 and 5.2, that are provided in the catheter body 100 outside of the isolated area defined by the isolating balloons 4 and functional opening 7. When catheter 100 is inserted into the lumen of a patient's hollow organ, the auxiliary holes 5.2 and 5.1 collectively provide a path for liquids and/or gaseous mediums to circumvent the isolated area defined by the isolating balloons 4 and functional opening 7. According to some embodiments, auxiliary holes 5.1 and 5.2 can serve as either inlets or outlets, respectively, depending on the orientation of the flow of liquid and/or gaseous mediums in the lumen of a patient's hollow organ. In other embodiments, for example as shown in FIG. 1A, no auxiliary holes 5.1 and 5.2 may be provided, and the flow of liquid and or gaseous medium around the isolated area 4 can be facilitated by first opening 9A and second opening 11A directly. It is to note that auxiliary holes 5.1, 5.2 allow a hollow organ contents, for example air, urine, blood, gastric or intestinal contents with inactive digestive ferments to pass to distal sections of a hollow organ without entering the area defined by the inflated isolating balloons 4 of the catheter 100, thereby preventing or eliminating the formation of congestive and/or inflammatory processes in patient's hollow organ that are caused, in particular, by accumulation of blood, urine, air, mucus and other contents in patient's hollow organ outside of the inflated isolating balloon 4 located farther or farthest from the distal end of the catheter 100.

According to some embodiments, catheter 100 as disclosed in either of FIG. 1A or FIG. 1B can facilitate bidirectional flow of fluid and/or gaseous mediums through elongate body of catheter 100 while bypassing the isolated area defined by isolating balloons 4 and the functional opening 7. For example, catheter 100 can be inserted into a lumen of a hollow organ wherein the flow of fluid and/or gaseous mediums through catheter 100 is oriented in a first direction (e.g., flowing distally from first opening 9A, 9B towards 11A, 11B and exiting catheter 100) or in a second direction (e.g., flowing proximally from second opening 11A, 11B towards opening 9A, 9B and exiting catheter 100). The bidirectional feature of catheter 100 can facilitate treatment of different hollow organs, different sections of hollow organs, and organs connected to hollow organs of a patient, such as, for example, the pancreas and liver. For example, when treating or diagnosing diseases of gastrointestinal tract, the above-referenced bidirectionality can allow for catheter 100 to be inserted endoscopically (e.g., through a patient's mouth or nose and through the esophagus of the patient) and allow for the flow of gaseous medium or fluid in a first direction, and the same catheter can also be inserted colonoscopically (e.g., through a patient's colon) and allow for the flow of gaseous medium or fluid in a second direction. It should be noted that catheter 100 is not limited to treatment and/or diagnosis of diseases of gastrointestinal tract, and can be used for diagnosis and/or treatment of diseases of any hollow organ or an organ connected to a hollow organ. Additionally, catheter 100 can be inserted not only endoscopically or colonoscopically, but in any other way known in the art, such as through a surgically made opening in a hollow organ of a patient. As another example, the bidirecitonality of the flow of fluid and/or gaseous mediums through the elongate body of the catheter 100 may be useful for facilitating treatment of a hollow organ such as a blood vessel. An area of the blood vessel may be isolated by the inflation of the isolating balloons while the main channel within the elongate body facilitates the bidirectional flow of fluid and/or gaseous medium (e.g., blood) through the blood vessel. The direction of the flow of a fluid (e.g., blood) through the main channel within the elongate body of the catheter 100 may be determined by the antegrade or retrograde way of inserting catheter 100 in relation to the normal flow of a fluid through the hollow organ (e.g., blood vessel). For example, catheter 100 could be inserted into an aorta either through femoral artery (retrogradely) or through an upper limb artery (anterogradely). Thus, bidirectionality of the flow enabled by the catheter 100 allows for an efficient flow of a fluid and/or gaseous medium through the main channel within the elongate body of the catheter 100 irrespective of the way it is inserted.

As shown in FIG. 1C, a full-body catheter 200 is disclosed. It should be noted that catheter 200 may have some or all of the features and functionalities as described with respect to catheter 100. As shown in FIG. 1C, catheter 200 has at its distal end an axial opening 11, the distal end being used for administering or inserting the catheter 200 into a patient followed by advancing the catheter 200 to a placement site within the lumen in a patient's hollow organ, for example, in the gastrointestinal tract, bile ducts, respiratory tracts, urinary system, patient vessels, a cavity related to uterine and vagina, etc. Furthermore, the catheter 200 is fitted with a three-way connector 20 at its proximal end opposite to the distal end of the catheter 200, wherein the proximal end is located outside of patient's body when the catheter 200 is inserted into the lumen of patient's hollow organ. In particular, when the catheter 200 is used for isolating the region in patient's duodenum, the catheter 200 is administered or inserted by its distal end into the patient's nasal passage followed by advancing the inserted catheter 200 along a patient duodenum to a placement site in patient's duodenum.

As shown in FIG. 1C, the three-way connector 21 in the catheter 200 may be a pipe or a tube provided with three branches or terminals: a central terminal 24, and two side terminals 22 and 26 hermetically isolated from the central terminal 24, wherein terminals 22, 24, 26 are each provided with a corresponding opening at their free end and each configured to connect to or join an appropriate functional appliance or device thereto.

The central terminal 24 positioned between the side terminals 22, 26 in the three-way connector 21 is configured to connect to or join an appropriate (first) functional appliance or device thereto, wherein the first functional device may be any device known in the art for supplying a gaseous medium or fluid (not shown), for example an enteral nutrition mixture. The device for supplying a gaseous medium or fluid as connected to the central terminal 24 may be, for example, an injection syringe filled, for example, with an enteral nutrition mixture to provide patient's organism with basic nutrients, energy, vitamins, macronutrients, micronutrients and/or etc., or a medical dropper filled, for example, with an enteral nutrition mixture, or other devices and appliances, including automated or semi-automated, suitable to connect the central terminal 24 thereto and supply a gaseous medium or fluid, for example an enteral nutrition mixture, to the central terminal 24. Therefore, the device for supplying a gaseous medium or fluid connected or attached to the central terminal 24 may be used to provide, for example, nutrition support or clinical nutrition to a patient when the catheter 200 is inserted within the lumen of duodenum (or other part of gastrointestinal tract). Furthermore, the device for supplying a liquid or gaseous medium, when connected or attached to the central terminal 24, may be used, for example, to sanitize the stomach and patient's duodenum.

The side terminal 22 being one of the two side terminals in the three-way connector 21 is designed to connect or attach to an appropriate (second) functional appliance or device thereto, wherein the second functional device may be implemented, for example, as a medical suction apparatus, an aspiration device or an aspirator (not shown) comprising of a storage reservoir or a storage container (not shown) for collecting biological gaseous medium, biological liquid and/or biological fluid and an air compressor (not shown) for aspirating or evacuating an air or another appropriate gaseous medium. The pressure for aspirating or evacuating the air or another appropriate gaseous medium may be adjusted by a one skilled in the art for corresponding hollow organs on the basis of information disclosed in, for example, in U.S. Pat. No. 6,712,798.

Furthermore, the second functional device to be attached to the side terminal 22 in the three-way connector 21 may be formed as a special device or device for supplying a gaseous medium or fluid (for example, medical products), for example an injection syringe filled with a liquid to be supplied, or a medical dropper filled with a liquid to be supplied, or other devices or appliances, including automated or semi-automated, suitable for connecting the side terminal 8 thereto and supplying said gaseous medium or fluid to the side terminal 22.

The other side terminal 26 in the three-way connector 21 is configured to connect or attach to an appropriate (third) functional appliance or device thereto, wherein the third functional device may be implemented as a special device or a device for supplying a liquid or gaseous medium under pressure (not shown), for example water or air, in particular an injection syringe filled with a liquid or gaseous medium (for example, water or air) to be supplied, or a medical dropper filled with a liquid (for example, water) to be supplied, or other devices and appliances, including automated or semi-automated, suitable for connecting the side terminal 26 thereto and supplying a fluid or gaseous medium to the side terminal 26.

It is to note that the above-described first functional device (not shown) connectable to the central terminal 24 of the catheter 200 for supplying a liquid, for example a enteral nutrition mixture, and the above-described second functional device (not shown) connectable to the side terminal 22 of the catheter 200 for sampling a biological fluid or biological gaseous medium or supplying a liquid or gaseous medium, and/or the third functional device (not shown) connectable to the side terminal 26 of the catheter 200 for supplying a liquid, for example water, in combination with the catheter 200 shown in FIG. 1C may form a system for isolating a region in a hollow organ of a mammal (not shown), which may be used for sampling a biological fluid or biological gaseous medium (for example, a biological liquid) being specific of certain hollow organ or organ connected to hollow organ of a mammal, or may constitute corresponding parts of such a system. In particular, the above system may be used for isolating a region in the duodenum of a mammal for sampling pancreatic juice and/or bile.

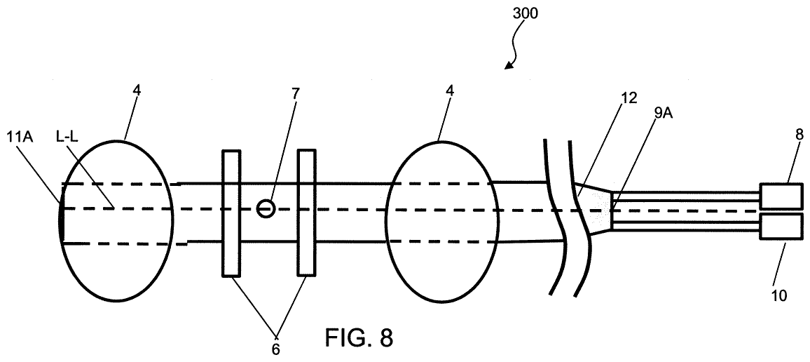

FIG. 2 illustrates a functional opening 7 provided in catheter 100, catheter 200, and/or catheter 300 (described with respect to FIG. 8, infra) according to aspects of the present disclosure. As shown in FIG. 2, catheter 100 and/or catheter 200 may include at least three channels within the elongate body of catheter 100 or catheter 200: Main channel 1, supply channel 2 for supplying a liquid or gaseous medium to the isolating balloons 4, and functional channel 3. Supply channel 2 and functional channel 3 may extend inside the wall of the elongate body of the catheter 100 and/or catheter 200 along the length of main channel 1. The main channel 1 may be in communication with first opening 9A, 9B and second opening 11A, 11B and may facilitate bidirectional flow of liquids and/or gaseous medium through the elongate body of catheter 100 and/or catheter 200. Supply channel 2 may be configured to supply a liquid or gaseous medium to the isolating balloons 4 in order to inflate the isolating balloons 4. Supply channel 2, main channel 1, and functional channel 3 may be hermetically sealed from one another. Supply channel 2 may be in fluid and/or gaseous communication with openings 13, 14, which connect supply channel 2 with an interior of isolating balloons 4, allowing isolating balloons 4 to be inflated when supply channel 2 is provided with a liquid or gaseous medium through proximal extension 10. According to some embodiments, instead of providing supply channel 2 for inflation/deflation of both isolating balloons 4, catheter 100 and/or catheter 200 may instead be provided with two supply channels (not shown) that are hermetically isolated from one another. A first supply channel may be in fluid and/or gaseous communication with opening 13, while a second supply channel may be in fluid and/or gaseous communication with opening 14, thereby facilitating the independent inflation and deflation of the isolating balloons 4. Functional channel 3 can be configured to collect gaseous medium, liquid and/or fluid, and/or aspirate or evacuating an air or another appropriate gaseous medium from the isolated area defined by the isolating balloons 4 when an appropriate functional device is attached to proximal extension 10.

When catheter 100 is inserted into the lumen of a patient's hollow organ, first opening 9A, 9B and second opening 11A, 11B are located within the patient's body. However, a central proximal extension (not shown) connected to first opening 9A, 9B, and in communication with main channel 1 may be connected to the above-described first functional device (not shown) to provide medical products, such as enteral or parenteral nutrition mixtures, which may be supplied under pressure from the first functional device to the first opening 9A, 9B, and into the main channel 1 to the interior of the hollow organ through the second opening 11A, 11B, as shown in FIGS. 1A-1B.

Supply channel 2 is configured to provide a gaseous or liquid medium to isolating balloons 4 through one of the corresponding outlets 13, 14 provided in the body of catheter 100 and/or catheter 200. As discussed above, according to some embodiments, instead of a single supply channel 2, catheter 100 and/or catheter 200 may be provided with a first supply channel and a second supply channel that are hermetically isolated from one another (not shown). The first supply channel may be configured to facilitate the inflation and/or deflation of a first isolating balloon 4 and the second supply channel may be configured to facilitate the inflation and/or deflation of a second isolating balloon 4. Supply channel 2 communicates with proximal extension 10 provided at proximal end of catheter 100. When catheter 100 is inserted into the lumen of a patient's hollow organ, proximal extension 10 is located outside of the patient's body and can communicate with an ambient atmosphere or environment outside of the patient's body. When the above described second functional device (not shown) is connected to proximal extension 10, a gaseous medium or fluid, for example air, water, or saline, may be supplied under pressure from the second functional device through proximal extension 10 and into supply channel 2, thereby causing the isolating balloons 4 to inflate through the corresponding holes 13, 14, provided in the elongate body of catheter 100 and/or catheter 200, causing isolating balloons 4 to be filled or inflated and thereby increase the size and/or volume of isolating balloons 4. The pressure used for filling or inflating the isolating balloons 4 with a fluid or gas for any particular hollow organ may be selected by one skilled in the art on the basis of information disclosed in, for example, in U.S. Pat. No. 7,722,568.

The inflation of the isolating balloons can be configured to result in a bilateral obstruction or blockage of the lumen of the patient's hollow organ, for example the lumen of a patient's duodenum where catheter 100 and/or catheter 200 is inserted, thereby allowing a part of the catheter 100 and/or catheter 200 defined by functional opening to be isolated between the isolating balloons 4 in the patient's hollow organ. Therefore, the isolating balloons 4, when inflated, allow a section of a patient's hollow organ, for example major and minor duodenal papillae of the duodenum, aneurysm, hollow organ's wall defect, etc., to be isolated from proximal and distal sections of the hollow organ, and therefore, excluding or preventing a biological liquid found in the isolated area defined by the isolating balloons 4 from mixing with other liquids and/or fluids, and allowing catheter 100 and/or catheter 200 to be fixed within the lumen of the patient's hollow organ due to the inflated isolating balloons 4 coming into contact with an inner wall surface of the hollow organ. In particular, in case when the catheter 100 and/or catheter 200 is inserted into the lumen of duodenum, the inflated isolating balloons 4 allow the minor duodenal papilla and/or the major duodenal papilla of patient's duodenum to be isolated from proximal and distal sections of the gastrointestinal tract and, therefore prevent mixing of the target biological liquid, wherein the target biological liquid may be a mixture of pancreatic juice and bile, with other biological liquids, such as gastric juice and intestinal contents. This ensures that digestive enzymes contained in pancreatic juice remain inactive.

In some embodiments consistent with the present disclosure, the isolating balloons 4 can be in communication through supply channel 2, thereby ensuring uniform redistribution of the gas or liquid used to expand or inflate the isolating balloons 4. For example, the isolating balloons 4 can undergo redistribution of the liquid or gaseous medium used for expansion during the passage of a peristaltic wave of the intestine or esophagus. In some embodiments consistent with the present disclosure, the isolating balloons 4 may be formed as self-inflating balloons inflated by air supplied to supply channel 2 of catheter 100 through the proximal extension 10, inflated by air supplied to supply channel 2 of catheter 200 through side terminal 26, or as a balloon inflated in any other way known in the art.

In one of the embodiments of the present invention, proximal extension 10 of catheter 100 and side terminal 26 of catheter 200 may be optionally equipped with a manually operated shut-off valve (not shown) to prevent the backflow or escape of a gaseous medium or fluid, in particular a gas or water located in the isolating balloons 4, the supply channel 2 and the proximal extension 10 of catheter 100 and/or the side terminal of catheter 200. It is to further note that the isolation valve may be used by a catheter user to adjust an inflation degree of the isolating balloons 4, wherein the user may visually control the inflation degree by using an endoscope. In particular, the user may reduce sizes or volumes of the inflated isolating balloons 4 by deflecting or venting a gaseous medium or fluid, in particular a gas or water, from the supply channel 4 by manually opening the check valve.

Functional channel 3 can extend along a part of the length of catheter 100 and/or catheter 200 and can communicate with proximal extension 8 of catheter 100 and/or side terminal 22 of catheter 200, wherein the functional channel is in communication with functional opening 7 on one end of functional channel 3 and is in communication with proximal extension 8 on the other end of functional channel 3 of catheter 100. For catheter 200, the functional channel may be in communication with functional opening 7 on one end of functional channel 3 and may be in communication with side terminal 22 of catheter 200. Accordingly, when the above-described third functional device (not shown) implemented as an aspirator is connected to proximal extension 8 of catheter 100, functional channel 3 will serve as an aspiration channel, and functional opening 7 will serve as an aspiration opening, wherein a negative air pressure or negative air medium pressure will be produced in an area of the interior of the hollow organ defined by the isolating balloons 4. The negative pressure (also referred to in the art as a technical vacuum) produced in the isolated interior of the patient's hollow organ promotes the evacuation or removal of a gaseous medium or fluid, for example, a biological fluid such as bile and pancreatic juice or pancreatic secretion, pus, blood, bronchial secretion, etc., to evacuate the gaseous medium or fluid from the isolated interior to the functional channel 3 through functional opening 7, and then from functional channel 3 through proximal extension 8, and finally from proximal extension 8 into a storage container of the aspirator associated with the above-described third functional device. It is to note that the functional channel 3 with a functional opening 7 may be used not only for sampling a gaseous medium or fluid being specific of a particular hollow organ or an organ connected to a hollow organ, but also for supplying a required fluid or gaseous medium, for example a drug in a liquid or gaseous form or coloring agent, to the isolated interior in case when the above-described third functional device (not shown) is implemented as a device for supplying a gaseous medium or fluid, for example a medical syringe or medical dropper, is connected to proximal extension 8. It is to note that when proximal extension 8 is used for administering therethrough a liquid or gaseous substance through functional channel 3 and entering the area defined by the isolating balloons 4 through functional opening 7, the administered substance, for example a drug or coloring agent, may affect the region isolated by the balloons 4 and have a diagnostic and/or therapeutic effect thereon.

As shown in FIG. 2, on an exterior of elongate body of catheter 100 and/or catheter 200 can be found two generally identical protrusions 6, each protrusion 6 may be formed as a ring-shaped projection or a ring having a radius which is greater than the radius of the elongate body of catheter 100 and/or catheter 200 and generally less than the radius of any one of the inflated isolating balloons 4. Each protrusion 6 may be located along a length of the elongate body of catheter 100 and/or catheter 200 and may be equally spaced in relation to the functional opening 7 between the isolating balloons 4. When catheter 100 and/or catheter 200 is inserted into the lumen of a patient's hollow organ, protrusions 6 may serve to space functional opening 7 from the inner wall of the hollow organ, thereby preventing functional opening 7 from being contacted by the mucosa of a patient's hollow organ when a technical vacuum is applied through functional opening 7, thereby preventing damage to the mucosal tissue of a hollow organ when technical vacuum is applied. In some embodiments consistent with the present disclosure, the protrusions 6 may have any other form different from a ring or ring-shaped form provided that such a form prevents the functional opening 7 from approaching the mucosa of a patient's hollow organ to allow functional opening 7 to provide a negative pressure to the isolated interior of the hollow organ without damaging the mucosal tissue of the hollow organ.

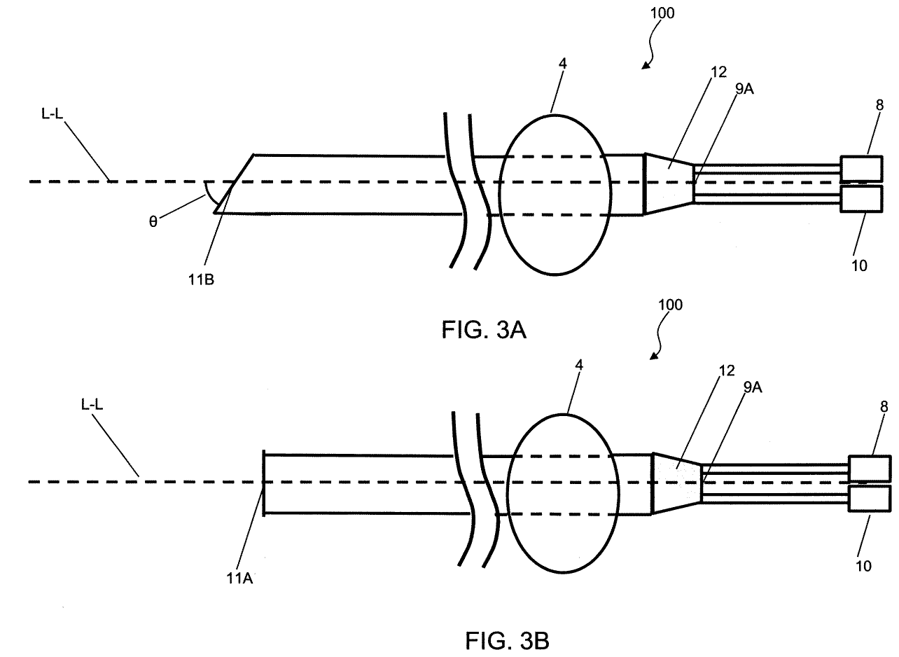

FIGS. 3A-3B illustrate catheter 100 having either an oblique (FIG. 3A) second opening 11B or a stub-shaped (FIG. 3B) second opening 11A, according to aspects consistent with the present disclosure. According to some embodiments, catheter 100 can be provided with a stub-shaped second opening 11A. In some embodiments, catheter 100 can be provided with an oblique second opening 11B. An oblique second opening 11B can provide the technical benefit of increasing the cross-sectional area of the second opening with respect to a stub-shaped second opening, thereby facilitating increased flow of fluids through the elongate body of catheter 100. According to some embodiments, the oblique second opening can form an angle θ in a range between approximately 15 degrees to approximately 65 degrees with respect to the longitudinal axis L-L defined by the elongate body of catheter 100. In a preferred embodiment the oblique second opening can form an angle θ of approximately 45 degrees with respect to the longitudinal axis L-L defined by the elongate body of catheter 100.

FIG. 4A illustrates a side profile view of catheter 100 having an oblique first opening 9B, according to aspects consistent with the present disclosure. According to some embodiments, catheter 100 can be provided with a stub-shaped first opening (e.g., as shown as 9A in FIGS. 1A, 1B, 3A, and 3B). In some embodiments, catheter 100 can be provided with an oblique first opening. An oblique first opening can provide the technical benefit of increasing the cross-sectional area of the first opening with respect to a stub-shaped first opening, thereby facilitating increased flow of fluids through the elongate body of catheter 100. A second technical effect provided by the oblique first opening can be facilitating atraumatic removal of the elongate body of catheter 100. According to some embodiments, the oblique first opening can form an angle φ in a range between approximately 15 degrees to approximately 65 degrees with respect to the horizontal axis N-N that runs parallel to longitudinal axis L-L that is defined by the elongate body of catheter 100. In a preferred embodiment the oblique first opening can form an angle φ of approximately 45 degrees with respect to the horizontal axis N-N. FIG. 4B illustrates a top-down view of catheter 100 having the oblique first opening, according to aspects consistent with the present disclosure. As can be seen in FIG. 4B, proximal extensions 8 and 10 exit oblique first opening 9B at the very proximal tip of first opening 9B. Positioning of proximal extensions 8 and 10 in such a way facilitates a more atraumatic removal of catheter 100 that is less likely to damage the inner tissue wall of a hollow organ.

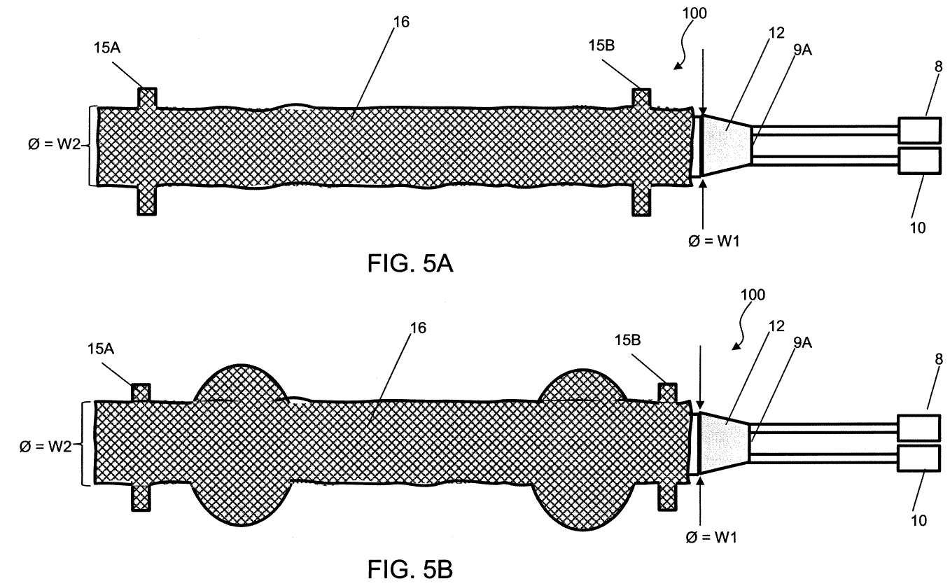

FIGS. 5A-5B illustrate a catheter 100 and/or catheter 200 with a net member with the isolating balloons in a deflated configuration (FIG. 5A) and with the isolating balloons in an inflated configuration (FIG. 5B), according to aspects of consistent with the present disclosure. As shown in FIG. 5A, catheter 100 and/or catheter 200 can be provided with a net member 16 that at least partly encloses the elongate body of catheter 100 and/or catheter 200 defined by the isolating balloons 4. According to some embodiments, the net member may be unattached to the elongate body of catheter 100 and/or catheter 200, but may remain secure around the elongate body of catheter 100 and/or catheter 200 due to net member 16 having a cross-sectional area at the ends of net member smaller than the cross-sectional area elongate body of catheter 100 and/or catheter 200. According to some embodiments, net member 16 may be constructed of an elastic or otherwise stretchable material, while in other embodiments, the net member may be constructed of a relatively inelastic material. A technical benefit of constructing net member 16 from an elastic material is the elasticity of net member 16 facilitates compression of isolating balloons 4 during deflation, facilitating the removal of catheter 100 and/or catheter 200 from a body cavity of a patient. Regardless of material, the net member may at least partly envelope or cover the isolated area defined by the isolating balloons 4. When the balloons 4 are inflated, as shown in FIG. 5B, the net member becomes strained or stretched, thereby moving or displacing the inner wall tissue in a hollow organ to a predetermined distance away from the functional opening 7. According to some embodiments, the predetermined distance may be determined in relation to the specific hollow organ or organ connected to the hollow organ that is being treated using the catheter 100 and/or catheter 200. In any case, the predetermined distance may be any distance sufficient to move or displace the inner wall of tissue from the functional opening 7 to allow functional opening 7 to provide a technical vacuum to the isolated area defined by the balloons 4 without interference from the inner wall of tissue.

As shown in FIG. 5A-5B, it should be noted that the width of the elongate body, W1, is smaller than the width of net member 16, referred to as W2. As net member is stretched between isolating balloons 4, net member 16 remains spaced away from the elongate body of catheter 100 and/or catheter 200. Accordingly, the width W2 of net member 16 facilitates the predetermined distance spacing of the elongate body of catheter 100 and/or catheter 200 from the inner wall of tissue, ensuring that functional opening 7 can provide a technical vacuum to the isolated area defined by the balloons 4 without interference from the inner wall of tissue. The net member 16, when in the strained state forms an outer casing which completely or at least partly encloses the catheter section defined by balloons 4 (e.g., between the isolating balloons 4), thereby completely or at least partly covering the functional opening 7. Net member 16 is configured to allow liquids and gaseous mediums to pass therethrough, thereby allowing the fluids specific to the hollow organ or organ connected to the hollow organ to penetrate or pass through a material of the net member 16 and to enter the functional opening 7. In particular, in the case when an aspirating device (not shown) is connected to proximal extension 8 of catheter 100 (or side terminal 22 of catheter 200) to provide, by the functional channel 3 and the functional opening 7 in the catheter 100 and/or catheter 200, a negative pressure in the isolated area between the isolating balloons 4, the stretched or strained net member 16 will hinder gluing or adhesion of the inner wall tissue in a hollow organ (for example, vessel walls, bowel mucosa, bronchus, stomach, ureter, or etc.) to the functional opening 7, thereby allowing the constant aspiration of biological material or biological fluid, for example, bile, pancreatic juice, bronchial secretions, etc., into the container of the aspirating device. According to some embodiments, the net member 16 can be attached to catheter 100 and/or catheter 200 distal to the distal isolating balloon 4. In some embodiments, the net member 16 can be attached to catheter 100 and/or catheter 200 both distal to the distal isolating balloon 4 and proximal of proximal isolating balloon 4 (e.g., in two locations). In some embodiments, the net member 16 can be attached directly to one or both isolating balloons 4 of catheter 100 and/or catheter 200. In some embodiments, the net member can be attached to catheter 100 and/or catheter 200 to one or more protrusion (e.g., protrusions 15A, 15B). Protrusions 15A, 15B can be provided on the elongate body of catheter 100 and/or catheter 200 outside of the area defined by isolating balloons 4. That is, protrusion 15A may be located distal to the distal isolating balloon 4 and protrusion 15B may be located proximal to proximal isolating balloon 4, as shown in FIG. 5B. Regardless of the attachment method of net member 16 to elongate body of catheter 100 and/or catheter 200, the stretched or strained net member 16 can hinder gluing or adhesion of the inner wall tissue in a hollow organ to the functional opening 7. According to some embodiments, net member may be formed as a net-like material secured in a strained state or at least partly strained state on protrusions 6 such that net member 16 completely or at least partly encloses or covers the functional opening 7.

In some embodiments, the net member 16 may include a taper at the distal end or may be fitted with a ring-like feature that has a smaller diameter than the diameter of the inflated balloons, thereby preventing the net member 16 from being removed from the elongate body of catheter 100 and/or catheter 200. The proximal end of the net member may be unattached to the elongate body of catheter 100 and/or catheter 200, and therefore may be movable along the shaft of the elongate body of the catheter proximal to the proximal balloon 4. However, the proximal end of the net member may also be tapered and/or fitted with a ring-like structure to prevent it from sliding over the proximal balloon 4 even when the balloons are in their deflated state.

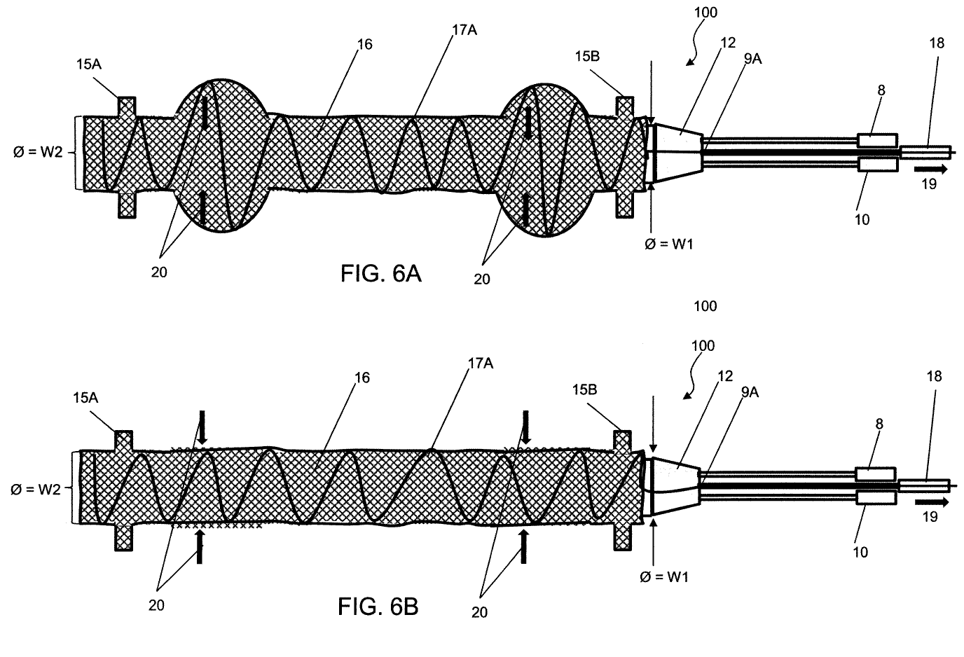

FIGS. 6A-6B illustrate a catheter 100 with a net member 16 and a compressive member 17A integrated into the net member 16, according to aspects of the present disclosure. In some embodiments, net member 16 can be provided with a compressive member 17A that can be woven or otherwise integrated into the material of net member 16. As shown and similarly described with respect to FIGS. 5A-5B, the width of the elongate body, W1, is smaller than the width of net member 16, referred to as W2. As net member is stretched between isolating balloons 4, net member 16 remains spaced away from the elongate body of catheter 100 and/or catheter 200. Accordingly, the width W2 of net member 16 facilitates the predetermined distance spacing of the elongate body of catheter 100 and/or catheter 200 from the inner wall of tissue, ensuring that functional opening 7 can provide a technical vacuum to the isolated area defined by the balloons 4 without interference from the inner wall of tissue.

Compressive member 17A can be provided as a thread or string of material that is integrated into net member 16 in a helical or “coil” fashion, as shown in FIGS. 6A-6B. According to some embodiments, the compressive member 17A can pass through an aperture or hole in elongate body of catheter 100 and/or catheter 200 (not shown), for example, such as one of auxiliary holes 5.2 as shown in FIG. 1B, and can extend out of first opening 9A, 9B via a third proximal extension 18 of catheter 100 or extend out of central channel 24 of catheter 200. An operator of catheter 100, such as a technician or physician, may pull compressive member 17A proximally as shown by arrow 19, thereby causing compressive member 17A to tighten circumferentially around net member 16, thereby causing isolating balloons 4 to become deflated by the compressive force applied by compressive member 17A in the radial direction indicated by arrows 20. According to some embodiments, the compressive member 17A provides the following technical effects/benefits to catheter 100: the compressive member 17A facilitates the deflation of the isolating balloons 4 prior to removal of the catheter 100 and/or catheter 200 from a patient, and the compressive member 17A reduces the potential for damage to tissues of the patient due to partially inflated balloons 4 interfering with the hollow organ's inner tissue wall when catheter 100 and/or catheter 200 is being removed from the hollow organ of a patient. It should be noted that the net member 16 and compressive member 17A may be implemented with the full-body catheter 200. For catheter 200, the compressive member may be attached to the net member 16 as described above, and can pass through an aperture or hole in elongate body of catheter 200, for example, such as one of auxiliary holes 5.2 as shown in FIG. 1C, and can extend out of the central channel 24. An operator of catheter 200 may pull the compressive member 17A proximally in a similar manner as described with respect to catheter 100 above, thereby causing compressive member 17A to tighten circumferentially around net member 16, thereby causing isolating balloons 4 to become deflated by the compressive force applied by compressive member 17A in a radial direction.

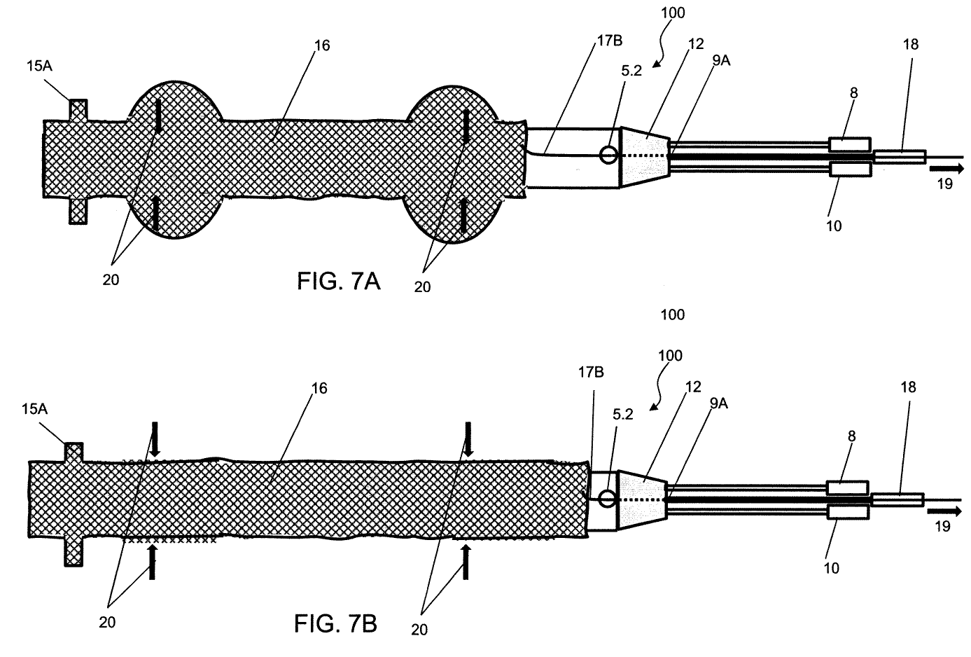

As shown in FIGS. 7A-7B, compressive member 17B may be attached at the proximal end of net member 16 rather than being integrated into the material of net member 16 in a helical fashion. In this simpler implementation, a proximal end of the compressive member 17B may be pulled proximally, thereby causing the net member 16 to stretch, causing the diameter of the net member to shrink as the compressive member is translated proximally. It can be seen in FIG. 7A that as the isolating balloons 4 are inflated, net member 16 is pulled distally towards distal end of elongate body due to the expansion of the isolating balloons 4. Similarly, as isolating balloons 4 are deflated, net member 16 is able to translate proximally towards the proximal end of catheter 100. As shown in FIGS. 7A-7B, the net member 16 may be attached to a point distal of the distal isolating balloon 4. For example, the net member 16 may be attached to a portion of the elongate body of catheter 100 (and/or catheter 200) or to protrusion 15A that secures net member 16 to the elongate body of catheter 100 (and/or catheter 200). In some embodiments, the net member 16 may be attached directly to the distal isolating balloon 4. In some embodiments, net member 16 is not attached at a distal end of catheter 100, but instead may be tapered such that net member 16 is prevented from sliding distally off the elongate body of catheter 100 (and/or catheter 200) and/or sliding proximally over the distal isolating balloon. A proximal end of net member 16 may not be attached to the elongate body of catheter 100 (and/or catheter 200) so that the proximal end of net member 16 is free to move along the elongate body of the catheter 100 and/or catheter 200 proximal of the proximal balloon 4. However, the proximal end of net member 16 may be similarly tapered such that net member 16 cannot slide distally over the proximal isolating balloon 4, even when the balloon is deflated. Compressive member 17B may be attached to the net member 16 at a point proximal of the proximal isolating balloon 4, and the compressive member 17B may extend into the interior of the elongate body through an aperture of catheter 100, such as through auxiliary hole 5.2, and can extend out of first opening 9A, 9B via a third proximal extension 18 of catheter 100 or extend out of central channel 24 of catheter 200. An operator of catheter 100 may pull compressive member 17B proximally as shown by arrow 19, thereby causing compressive member 17B to stretch the net member 16, thereby causing a diameter of net member 16 to contract in the radial direction, as shown by arrows 20. The compressive member 17B provides the technical effect of facilitating deflation of the isolating balloons 4 prior to removal of catheter 100 and/or catheter 200 from a patient, and the compressive member 17B reduces the potential for damage to tissues of the patient due to partially inflated balloons 4 interfering with the hollow organ's inner tissue wall when catheter 100 and/or catheter 200 is being removed from the hollow organ of a patient. The simpler implementation of the compressive member 17B and net member 16 may be used with either stent-like catheter 100 and/or full-body catheter 200. For catheter 200, the compressive member 17B may be attached to the net member 16 as described above, and can pass through an aperture or hole in elongate body of catheter 200, for example, auxiliary hole 5.2 as shown in FIGS. 7A-7B, and can extend out of the central channel 24. An operator of catheter 200 may pull the compressive member 17B proximally in a similar manner as described with respect to catheter 100 above, thereby causing compressive member 17B to stretch net member 16, thereby causing isolating balloons 4 to become deflated by the compressive force applied by the stretched net member 16 in a radial direction as indicated by arrows 20.

FIG. 8 illustrates catheter 300 for isolating a region in a hollow organ according to aspects of the present disclosure. Catheter 300 may may have one or more of the same features of catheter 100, and may be used in any manner as described with respect to catheter 100. More particularly, net member 16 and compressive members 17A, 17B may be implemented with catheter 300 as described with respect to FIGS. 6A, 6B, 7A, and 7B. Catheter 300 may have a shortened elongate body on the distal end of catheter 300. More specifically, second opening 11A on the distal end of the elongate body may be located approximate the distal isolating balloon 4. Otherwise, catheter 300 may have some or all of the features of catheter 100. For example, catheter 300 may include first opening 9A, a plurality of proximal extensions 8, 10, a tapered section 12 of elongate body, isolating balloons 4, second opening 11A, functional opening 7, and two projections 6 on either side of functional opening 7. It should be noted that functional opening 7 as depicted and described with respect to FIG. 2, is applicable to catheter 300. That is, catheter 100 may include at least three channels within elongate body of catheter 300—Main channel 1, supply channel 2 for supplying a liquid or gaseous medium to isolating balloons 4, and functional channel 3. Supply channel 2 and functional channel 3 may extend inside the wall of the elongate body of the catheter 300 along the length of main channel 1. The main channel 1 may be in communication with first opening 9A, 9B and second opening 11A, 11B and may facilitate bidirectional flow of liquids and/or gaseous medium through the elongate body of catheter 300. Supply channel 2 may be configured to supply a liquid or gaseous medium to the isolating balloons 4 in order to inflate the isolating balloons 4. Supply channel 2, main channel 1, and functional channel 3 may be hermetically sealed from one another. Supply channel 2 may be in fluid and/or gaseous communication with openings 13, 14, which connect supply channel 2 with an interior of isolating balloons 4, allowing isolating balloons 4 to be inflated when supply channel 2 is provided with a liquid or gaseous medium through proximal extension 10. According to some embodiments, instead of providing supply channel 2 for inflation/deflation of both isolating balloons 4, catheter 300 may instead be provided with two supply channels (not shown) that are hermetically isolated from one another. A first supply channel may be in fluid and/or gaseous communication with opening 13, while a second supply channel may be in fluid and/or gaseous communication with opening 14, thereby facilitating the independent inflation and deflation of the isolating balloons 4. Functional channel 3 can be configured to collect gaseous medium, liquid and/or fluid, and/or aspirate or evacuating an air or another appropriate gaseous medium from the isolated area defined by the isolating balloons 4 when an appropriate functional device is attached to proximal extension 10.

Stent-like catheter 300 may provide some technical benefits in vascular applications over stent-like catheter 100 due to the shortened elongate body on the distal end of catheter 300. For example, catheter 300 may be advantageous when inserting catheter 300 into tortuous vasculature of a patient, and may facilitate removal and withdrawal of catheter 300 from the vasculature of a patient as compared to catheter 100.

Depending on the use of the catheter 100, catheter 200, and/or catheter 300 according to the present invention and on anatomical parameters of the hollow organ, a length and diameter of the catheter 100, catheter 200, and/or catheter 300, a wall thickness of the catheter 100, catheter 200, and/or catheter 300, a location and diameter of the isolating balloons 4 in the catheter 100, catheter 200, and/or catheter 300, and the location of the holes may vary. One skilled in the art may easily adjust any parameter of the catheter 100, catheter 200, and/or catheter 300 depending on the dimensions of a particular hollow organ.

The thickness of the channels of the catheter 100, catheter 200, and/or catheter 300 and the size of the isolating balloons 4 may be adjusted by one skilled in the art on the basis of information disclosed in the art, for example, U.S. Pat. Nos. 9,526,874, 6,692,465, 5,843,050, 5,919,163, international publication WO 2009/035581, U.S. Pat. Nos. 5,397,305, 8,398,589, 7,722,568, 6,712,798, 6,638,245, 1,009,865 and/or other information sources.

The length of the catheter 100, catheter 200, and/or catheter 300, a distance between the isolating balloons 4, and a distance from the isolating balloons 4 to the distal end may be matched by one skilled in the art on the basis of information disclosed in the art, for example, U.S. Pat. Nos. 5,314,409, 5,658,264, US patent application No. 20150150572, U.S. Pat. Nos. 5,843,050, 5,397,305, 7,070,606, 6,712,798, 1,009,865 and/or other information sources.

For example, in one of the preferable embodiments of the present invention, the catheter 100, catheter 200, and/or catheter 300 may be implemented as a pancreato-digestive catheter and may have a length of 130 cm. In the present embodiment of the present invention, the isolating balloons 4 are correspondingly spaced at 10 cm and 25 cm from the distal end of the catheter 100, catheter 200, and/or catheter 300, the distal end being used for inserting the catheter 100, catheter 200, and/or catheter 300 into the gastric cavity and the lumen of patient's duodenum, so that expanding or inflating of the isolating balloons 4 allows for isolation of a duodenum area having a length of at approximately 15 cm. In the present embodiment of the present invention, the functional opening 7 may be in the middle between the isolating balloons 4, i.e., at a distance of approximately 7.5 cm from each of the isolating balloons 4 and at a distance of approximately 17.5 cm from the distal end of the catheter 100, catheter 200, and/or catheter 300.

For the treatment of vascular pathology (e.g., blood vessel injury) the catheter 100 and/or catheter 300 may be implemented with elongate body with a length of 15-20 cm and proximal extensions with a length of 100-200 cm, depending on the target location where catheter 100 is to be placed. The distance between isolating balloons 4 may be 5-10 cm and the distal end of the elongate body of catheter 100 may or may not extend beyond the distal balloon 4. The functional opening 7 may be located 2.5-5 cm from each of the balloons 4.

Use of the catheter 100, catheter 200, and/or catheter 300 according to the present invention for isolating a region in patient's hollow organ is illustratively described above in relation to patient's duodenum. However, the use of the catheter 100, catheter 200, and/or catheter 300 is not limited by patient's duodenum. Therefore, it is clear for one skilled in the art that the catheter 100, catheter 200, and/or catheter 300 according to any of the above-described embodiments can be similarly inserted into a lumen of any another hollow (tubular) internal organ of a patient, in particular mammals, for example in the esophagus, stomach, duodenum, small intestine, large intestine, respiratory tracts, urinary tracts (urogenital system tracts), veins, arteries, vagina, uterus, uterine (Fallopian) tubes, vertebral canal, or any appropriate internal tubular organ of a patient, the patient tubular organ being related to a corresponding functional system (apparatus of organs) of a mammal organism from a group of systems including: digestive system, respiratory system, urinary and reproductive systems (combined into the genitourinary system or urogenital system), endocrine system, circulatory system and immune system, and skeletal system.

Therefore, when used, the catheter 100, catheter 200, and/or catheter 300 according to the present invention may be inserted by the user into the lumen of a patient's duodenum under control of an endoscope (not shown) or of a radiographic equipment (for example, a fluoroscopy equipment) such that one of the corresponding isolating balloons 4, the farthest from the proximal end of the catheter 100, catheter 200, and/or catheter 300, is located in the bulb of patient's duodenum, wherein the endoscope may be manipulated by the user or endoscopist assisting the user. In particular, it is to note that the insertion process of the catheter 100, catheter 200, and/or catheter 300 into the lumen of patient's duodenum, the removal process of the catheter 100, catheter 200, and/or catheter 300 from the lumen of patient's duodenum after sampling a required amount of a biological liquid and the aspiration process are atraumatic and do not depend on the anatomical features of both the patient and neoplasms. It is to note that the isolating balloons 4 are in deflated state when the catheter 100, catheter 200, and/or catheter 300 is inserted by the user into the lumen of patient's duodenum.

According to one example, the catheter 100, catheter 200, and/or catheter 300 may be preliminary equipped with at least one loop designed to grip it with biopsy forceps. To insert the catheter 100, catheter 200, and/or catheter 300 into the desired placement site in patient's duodenum the distal end of the catheter 100, catheter 200, and/or catheter 300, well lubricated with Vaseline oil, is administered through the nasal passage and advanced to the gastric cavity; then the endoscope is further administered or advanced in parallel with the catheter 100, catheter 200, and/or catheter 300 through the patient oral cavity to the patient's gastric cavity to capture, by means of the endoscope biopsy forceps, the loop(s) of the catheter 100, catheter 200, and/or catheter 300; finally the captured catheter 100, catheter 200, and/or catheter 300 is guided or advanced along with the endoscope to patient's duodenum. Subsequently, under control of an endoscope, one of the corresponding isolating balloons 4 of the catheter 100, catheter 200, and/or catheter 300, the farthest from the proximal end of the catheter 100, catheter 200, and/or catheter 300, is placed in the bulb of patient's duodenum.

According to another example, a metal guidewire may be preliminary administered or inserted into the main channel 1 of the catheter 100, catheter 200, and/or catheter 300. To insert the catheter 100, catheter 200, and/or catheter 300 into a desired placement site in patient's duodenum the distal end of the catheter 100, catheter 200, and/or catheter 300, well lubricated with Vaseline oil, is inserted through the nasal passage and then advanced to the gastric cavity by using the metal guidewire of the catheter 100, catheter 200, and/or catheter 300; then the endoscope is inserted or advanced in parallel with the catheter 100, catheter 200, and/or catheter 300 through the patient oral cavity to the patient gastric cavity to capture, by means of the endoscope biopsy forceps, a first ligature upon detection of the distal end of the catheter 100, catheter 200, and/or catheter 300 and to press the catheter 100, catheter 200, and/or catheter 300 to the endoscope by pulling up the catheter 100, catheter 200, and/or catheter 300 by using the captured first ligature. Subsequently, the endoscope and the catheter 100, catheter 200, and/or catheter 300 pressed against the endoscope are guided through the pylorus to the patient's duodenum, and one of the corresponding isolating balloons 4 of the catheter 100, catheter 200, and/or catheter 300, the farthest from the proximal end of the catheter 100, catheter 200, and/or catheter 300, is placed under control of the endoscope within the bulb of patient's duodenum.

Then, the user connects or attaches the above-described second functional device (not shown) to proximal extension 10 of catheter 100 and/or catheter 300 (or side terminal 22 of catheter 200), when the second functional device is intended to supply a liquid or gaseous medium, for example a gas or water, to the supply channel 2 of the catheter 100, catheter 200, and/or catheter 300 in a required amount or volume, in particular water in a volume of 40-70 ml, to allow the expansion and inflation of the isolating balloons 4 to closely adjoin to a mucosa of the duodenum, thereby isolating required area of patient's duodenum containing the major duodenal papilla and the minor duodenal papilla, in particular preventing gastric contents and/or duodenal contents with inactive digestive ferments from entering the isolated area. In particular, it is to note that results experimentally obtained by the inventors show that 40-70 ml of air is required to sufficiently inflate the isolating balloons 4 to a required size for bilateral obturation or occlusion of patient's duodenum lumen where the catheter 100, catheter 200, and/or catheter 300 may be inserted, as an illustrative example.

Then, the user joins or connects the above-described third functional device (not shown) implemented as an aspirator to the functional channel 3, wherein aspirator is preliminary configured for a constant or variable operation mode providing a required working pressure depending on a particular task. The aspirator activated by the user allows an air medium or air to be sucked, through the functional opening 7 and the functional channel 3 communicating therewith, from the isolated interior of the patient duodenum, the isolated interior corresponding to the periampullary zone of the duodenum, thereby producing in the interior negative pressure corresponding to pressure normally created by the peristalsis of the duodenum, in particular a negative pressure of 40-100 mm wg (9-14 kPa)). The negative pressure provided in the isolated interior of patient's duodenum allows a biological liquid in the form of pancreatic juice or pancreas secretion in combination with bile to be evacuated or retrieved from the pancreas through the major duodenal papilla and the minor duodenal papilla, thereby removing said biological liquid, through the functional opening 7 and the functional channel 3, from the isolated area of patient's duodenum and collecting it in a storage container (not shown) of the aspirator. In some cases, there may be further collected in the container a mucosal secretion of the bile and pancreatic ducts, the mucosal secretion being one of the varieties of the biological liquid being specific of duodenum, and/or some biological contents which would enter the bile and the pancreatic ducts as a result of reflux and would contain liquids produced by the organism, the liquids being specific of human duodenum. Subsequently, the biological liquid collected in the storage container of the functional device (not shown), can be delivered for cytological examination and/or molecular genetic examination or other analyses to evaluate characteristics of a morphological structure of cellular elements of pancreatic secretion, cell elements allowing one to detect or reveal pathologic processes being specific, for example, of intraductal neoplasms, neuroendocrine tumors or pancreatic cancer, and to detect neoplasms at an early stage of development, and to perform differential diagnosis between different types of tumors by determining expressions of specific markers (NKX2, S100P, CEA, EFR3A/B, MUC1, MUC2, MUC5, ANXA1, A2, KRT7, MMP7, MMP9, IGFBP3, PSCA, PRSS2, SHh, KRas, TP53, SMAD4, BRCA1, miRNA 21, and miRNA 155).

Furthermore, after connecting the aspirator to the functional channel 3 or instead of the aspirator, a device for supplying a gaseous medium or fluid (for example, an injection syringe or a medical dropper) may be connected to the functional channel 3, enabling the delivery of a required fluid or gaseous medium, for example a drug in a liquid or gaseous form, to the isolated interior of patient's duodenum.

It is to note that the catheter 100, catheter 200, and/or catheter 300 according to the present invention may remain in the inserted state for a sufficiently long period of time, for example up to seven days (i.e., up to 168 hours), allowing for the pancreatic secretion to be collected in an appropriate amount for reliable identification and verification of pathologic tumor material, so that it generally improves the efficiency of diagnosis and, therefore, subsequent treatment of pancreatic cancer. Furthermore, prolonged sampling increases the likelihood of detecting pathologic cells and other biomarkers not only for pancreatic cancers, but also for malignant conditions of bile ducts and liver, including cholangiocarcinomas and/or hepatocellular cancer.

It is to further note that stimulation of the secretion of pancreatic juice and bile is not required when the catheter 100, catheter 200, and/or catheter 300 is used, so that biological material collected has true biochemical and physiological profile presenting true functional state of pancreas, liver, bile ducts, gallbladder.

If necessary, the excretory function of pancreas may be evaluated by examining a biochemical composition of pancreatic secret collected in the above-described manner in the storage container (not shown) in combination with bile by using the catheter 100, catheter 200, and/or catheter 300 according to the present invention.

Furthermore, a qualitative and quantitative analysis of pancreatic juice and bile allows for a true biochemical profile of both biological materials and a volume of daily secretion to be evaluated, thereby diagnosing functional condition of an organ.

The catheter 100, catheter 200, and/or catheter 300 according to any one of the above-described embodiments of the present invention may be used for diagnosing or monitoring the development or monitoring the treatment of at least one disease selected from a group comprising: bleeding, haemostasis, gastritis, acute and chronic pancreatitis, autoimmune pancreatitis pancreatic neoplasms and cancer, bile duct cancer, cholangiocarcinoma, hepatocellular cancer, cholangitis, cholelithiasis, defects of a hollow organ wall, fistula of hollow organs, autoimmune hepatitis, infectious hepatitis, sclerotic cholangitis, Crohn disease, ulcerative colitis, eosinophilic esophagitis, aneurismal or diverticular protrusions of a hollow organ wall, bleeding of hollow organs, bleeding into hollow organs, strictures of hollow organs, esophagus neuromuscular disorders, stomach neuromuscular disorders, duodenum neuromuscular disorders, neuromuscular disorders of the small intestine and colon and rectum, neuromuscular disorders of bile ducts, neuromuscular disorders of urinoexcretory ways, and neuromuscular disorders of respiratory tracts, vascular thrombosis and embolization, vessels' malformations.

Furthermore, the catheter 100, catheter 200, and/or catheter 300 according to any one of the above-described embodiments of the present invention may be used for treating at least one disease selected from a group comprising: bleeding, pancreatitis, cholangitis, gastrointestinal wall defects, fistulas of hollow organs, vascular defects, vascular aneurisms, vascular thrombosis and embolization, defects of a hollow organ wall, bleeding of hollow organs, bleeding into hollow organs, gastrointestinal hemorrhages, bleeding from the windpipe (trachea), bronchial bleeding, pulmonary hemorrhage, uterine bleeding, vessels' malformations, Crohn disease, ulcerative colitis, eosinophilic esophagitis, malignant diseases that occur in hollow organs and organs that are connected to hollow organs.

Furthermore, the catheter 100, catheter 200, and/or catheter 300 according to any one of the above-described embodiments of the present invention may be used to isolate an area of a hollow organ when performing a surgical procedure or performing surgical manipulations, in particular in relation to tissues of the wall of a hollow organ. In an embodiment, the hollow organ to be isolated may be a vessel, such as a blood vessel.

In particular, there is a treatment or therapeutic effect achieved by restoring and facilitating the evacuation of bile or of pancreatic juice from the corresponding ducts into duodenum when the catheter 100, catheter 200, and/or catheter 300 is used. The pathogenesis of acute inflammatory diseases, such as pancreatitis and cholangitis, is caused at least in part by the impairment or disruption of the outflow of pancreatic juice and bile, the outflow impairment being caused, in particular, by impaired motility (peristalsis) of duodenum of a mammal, so that isolated area with a controllable negative pressure (for example, 40-100 mm wg) producing aspiration of pancreatic juice and bile by using the catheter 100, catheter 200, and/or catheter 300 allows one to address this medical problem and, therefore, to contribute to the treatment of the above acute diseases.

Furthermore, when the catheter 100, catheter 200, and/or catheter 300 is used, there is a treatment or therapeutic effect achieved by evacuating contents from a region pertaining to a wall defect of gastrointestinal tract, so that the isolated region with a controllable negative pressure (for example, at level 70-100 mm wg), allows one to evacuate all of the biological liquids from a defect area and solve this medical problem and, therefore, to contribute to solving said medical problem of a patient.

It is clear for one skilled in the art that the catheter 100, catheter 200, and/or catheter 300 according to the present invention may be made of any suitable materials on the basis of information disclosed in, for example, U.S. Pat. No. 7,722,568 or 6,638,245, wherein the catheter 100 may be preferably made of polymeric materials.

### EXAMPLES

Example No.1. A patient No.1 was admitted with girdle pain in the upper abdomen. Amylase in a blood was 1150 U/L. Results of ultrasound examination were as follows: pancreas was increased in size; the head was 35 mm; the body was 32 mm; the tail was 21 mm; strongly heterogeneous; the contour was blurred; the parapancreatic tissue was edematous; liquids were not detected. The MSCT picture corresponds to acute hydropic pancreatitis. The patient started a therapy according to international guidelines IAP/APA 2013, wherein the catheter 200 according to the present invention was inserted into the patient duodenum for 24 hours, and active aspiration method was performed. After 24 hours, the level of amylase in a blood dropped to 230 U/L, and the pain syndrome was managed.
Example No.2. A patient No.2 was admitted with a clinical picture of obstructive jaundice. The examination revealed an increased level of leukocytes up to 14.1×10\hat{ }9, wherein total bilirubin was 145 mMol/L, and direct bilirubin was 113.2 mMol/L. Amylase in a blood was 57 U/L, alanine aminotransferase (ALT) was 391 U/L, and aspartate aminotransferase (AST) was 90 U/L. Results of ultrasound were as follows: multiple calculi up to 1.3 cm in diameter were detected in the gallbladder, and the bile ducts were dilated up to 11 mm. Results of magnetic resonance imaging (MRI) were as follows: a shadow corresponding to a calculus of 6 mm was detected in the distal section of the common bile duct. Results of the endoscopic retrograde cholangiopancreatography (ERCP) were as follows: a single 8 mm calculus was identified, and then it was endoscopically removed. To prevent the acute pancreatitis the catheter 200 according to the present invention was inserted into patient's duodenum for 24 hours, and active aspiration method was performed. In the postoperative period, the level of amylase in a blood did not increase more than 87 U/L.
Example No.3. A patient No.3 was admitted in a planned manner for examination and treatment. Previously performed MSCT detected formation of an anomalous body in pancreas. Endoscopic ultrasound detected formation of an anomalous body having the size of 2 cm by 3 cm, wherein the fine-needle biopsy was technically impossible. To selectively sample pancreatic juice, the catheter 200 according to the present invention was inserted into patient's duodenum. Adenocarcinoma cells and mutations in driver genes, exosomes and protein markers were detected in pancreatic juice obtained thanks to cytological and molecular examinations.
Example No.4. A patient No.4 was admitted with a clinical picture of obstructive jaundice. Results of MRI: a block at the level of confluence of the lobar right and left hepatic ducts was revealed, wherein the block corresponds to the Klatskin tumor of type IIA. The catheter 200 for isolating a region in a hollow organ according to the present invention was inserted into patient's duodenum, and active aspiration was performed. Abnormal cells and markers of malignant neoplasms were detected in the obtained bile thanks to cytological and molecular examinations.
Example No.5. Patient No.5 was diagnosed with chronic pancreatitis. Despite the gastroenterologist's therapy and prescribed enzymatic drugs taken by the patient, body mass reduction, defecation disorders, and diarrhea were observed. To evaluate the functional state of pancreas the catheter 200 for isolating a region in a hollow organ according to the present invention was inserted into patient's duodenum for 24 hours, and active aspiration was performed. As a result of a biochemical test performed on pancreatic juice obtained, decreased levels of alpha amylase and lipase were revealed, and also a decrease in the daily excretion of pancreatic juice and bile was revealed. As a result, the dose of enzymatic drugs taken by the patient was increased, and ursodeoxycholic acid drugs and bile substitutes were additionally prescribed.
Example No.6. A patient No.6 with jaundice was admitted to an infectious diseases clinic in the city. Results of physical examination showed an enlarged liver, wherein results of a biochemical blood test showed the following: total bilirubin was 329 mMol/L, direct bilirubin was 141 mMol/L, ALT was 1040 U/L, AST was 804 U/L. Serological analysis of blood for HCV antigens, HBsAg was negative. The catheter 200 for isolating a region in a hollow organ was inserted into the patient, wherein 100 ml of bile was obtained as a result of active aspiration, and a high copy number of the HCV virus was detected by PCR analysis of bile.
Example No.7. A patient No.7 was treated in an infectious diseases clinic for the verified viral hepatitis. After the course of interferons, a repeated PCR analysis of blood did not reveal an active viral process. The catheter 200 for isolating a region in a hollow organ was inserted into the patient, wherein 200 ml of bile was obtained as a result of active aspiration, and the PCR analysis of the bile obtained showed an ongoing viral process, so that the scheme and duration of therapy were changed.
Example No.8. A patient No.8 was diagnosed with intestinal fistula and peritonitis. The operation, sanitation and drainage of the abdominal cavity were performed on an emergency basis. In order to isolate an intestinal wall defect, reduce its contact with intestinal contents, improve the healing process, and also to provide an additional modality for treating peritonitis by separating abdominal cavity and pathological substrate, the catheter 200 for isolating a region in a hollow organ according to the present invention was inserted into an injury area of an intestine, such that the isolating balloons 4 were located above and below the defect. After inflating the balloons, the isolated area was created, and another channel was connected to a suction apparatus to provide sanitization of the area of intestinal fistula to facilitate defect healing. Enteral nutrition was also administered to the patient through the catheter 200.
Example No.9. A patient No.9 with girdle pain in the abdomen was admitted to the hospital as an emergency. Based on the results of examination, the patient was diagnosed with acute pancreatitis, of moderate severity, according to the Atlanta classification. At the time of admission, the amylase level was 1320 U/L. The catheter 200 for isolating a region in a hollow organ according to the present invention was inserted into the patient, wherein the functional channel was connected to a pump for producing a controllable negative pressure, while enteral nutrition and medical products were administered to the patients through the same catheter 200. As a result, the dynamics of biochemical parameters became positive in accordance with the BISAP scale within 24 hours, and the patient recovered after 7 days.
Example No.10. A patient No.10 was admitted to the hospital as an emergency with pain in the right hypochondrium, hectic form of fever and periodic yellowing of the skin. The anamnesis showed that 3 months before the current hospitalization the patient underwent endoscopic manipulation in relation to the bile ducts in order to remove calculus from them. Ultrasound, MSCT showed no changes in the liver, ducts or the presence of abscesses. In order to diagnose cholangitis, the catheter 200 for isolating a region in a hollow organ was inserted into the patient, and 50.0 ml of bile was sampled and subjected to bacteriological examination. As a result, the abundant growth of Klebsiella spp. was revealed, and its sensitivity to antibacterial drugs was determined. After the selection of pathognomonic antibiotic therapy, the cholangitis symptoms disappeared, and the patient was discharged for an outpatient treatment.
Example No.11. Patient No.11 underwent sigmoid colon resection due to the presence of a mass lesion. It was revealed during intestine mobilization that a formation invaded all the layers of intestine, wherein there was regional lymphadenopathy with involvement of retroperitoneal tissue. When mobilizing the intestine with a tumor, communication was established with the injured ureter of the left kidney. Urologists were called in the operating room and treated surgically the defect of the ureter. To prevent complications, an adjusted in size catheter 200 for isolating a region in a hollow organ according to the present invention was administered retrogradely along a guide wire, and one of the balloons was inflated in the pelvis of the left kidney, and the other balloon (the lower balloon) was inflated in the bladder, wherein the functional channel was connected to a suction apparatus for producing a controllable negative pressure. The catheter 200 was removed in 7 days, wherein the excretory function of the left kidney was not impaired. The patient was discharged for outpatient treatment. It should be noted that catheter 100 and/or catheter 300 may have been utilized in lieu of catheter 200 in this example treatment.
Example No.12. A patient No.12 with a pulmonary hemorrhage was admitted. Bronchoscopy revealed a putrescent bleeding tumor of the right main bronchus. For the purpose of hemostasis and preservation of bronchial patency, and also for the prevention of lung atelectasis, catheter 100 for isolating a region in a hollow organ according to the present invention was administered into the right bronchus. The catheter 100 was inserted such that the tumor was between the balloons, wherein inflation of the balloons stopped the blood flowing to other parts of the bronchus. To stop the bleeding a hemostatic agent was administered through the functional channel of the catheter 100. As a result, there was no relapse of the bleeding for three (3) days of observation. The catheter 100 was subsequently removed. The patient was discharged for outpatient treatment ten (10) days later. It should be noted that catheter 200 and/or catheter 300 may have been utilized in lieu of catheter 100 in this example treatment.
Example No.13. A patient No.13 with an instrumentally confirmed clinic of mediasthenitis was admitted on an emergency basis. The examination revealed an esophagus defect due to necrosis caused by a piece of meat. For treating the esophagus, the catheter 200 for isolating a region in a hollow organ according to the present invention was inserted. The balloons of the catheter 200 were inflated to isolate the defect, and the functional channel was connected to a suction apparatus to provide a controllable negative pressure. As a result, the patient was discharged for outpatient treatment after 1.5 months.
Example No.14. A patient No.14 with headaches and general cerebral symptoms was admitted on an emergency basis. MSCT with intravenous contrast enhancement revealed an aneurysm in the basin of the right middle cerebral artery with signs of extrusion. The patient was taken to the angiographic operating room, wherein the catheter 100 for isolating a region in a hollow organ according to the present invention was administered through the femoral approach. Positioning of the catheter 100 was performed under control of fluoroscopy such that the balloons isolated the area of the aneurysm, wherein the functional channel was opposite the aneurysm. A filling solution filling the aneurysm cavity was administered through the functional channel. After the necessary exposure, the catheter 100 allowed to maintain a circumferential blood flow. Subsequently the catheter 100 was removed. The patient was discharged from a hospital in 21 days.
Example No.15. A patient No.15 was admitted in a planned manner for surgical treatment of pancreatic cancer. An examination showed a pancreatic head tumor invading the inferior vena cava. To reduce blood loss the catheter 100 for isolating a region in a hollow organ according to the present invention was inserted into the vein, and the balloons were inflated above and below the pancreas head tumor, thereby shunting blood and returning blood to the heart. During the resection of the gland, a section of the inferior vena cava was resected with autoplasty of the saphenous vein of the lower extremity. The blood loss was 500 ml. Therefore, optimal temporary and physiological conditions were created both for the patient and surgeons.
Example No.16. A patient No.16 was admitted for surgical treatment of sigmoid colon cancer. In the postoperative period, the patient developed colo-rectoanastomosis failure for ⅓ of the circumference. To treat the colon the catheter 200 for isolating a region in a hollow organ according to the present invention was retrogradely inserted into the rectum such that one of the balloons of the catheter 200 was positioned most proximally in relation to the defect, and the other balloon of the catheter 200 was positioned most distally in relation to the defect. When the balloons of the catheter 200 were inflated, area of the defect was isolated from other sections of the large intestine. Through to the main channel, proximal sections of the bowel were cleaned with water. As a result, the defect was closed in 3 weeks.
Example No.17. A patient No.17 was admitted with suspected liver tumor. To perform a liquid biopsy the catheter 200 for isolating a region in a hollow organ according to the present invention was inserted; an area related to the major and minor duodenal papillas was isolated by the balloons of the catheter 200, and aspiration of bile started. When the catheter 200 was removed, a bile aspirate was delivered for genetic and cytological examination, wherein no pathologic markers and cells were detected. When the bile is taken, the stocking covering the balloons of the catheter 200 was removed and washed with a buffer solution, whereupon the water was collected, and after centrifugation a cell pellet was obtained. Cytological examination of the obtained cell sediment revealed the presence of hepatocellular cancer cells.
Example No.18. A patient No.18 was admitted on an emergency basis with a uterine bleeding. Colposcopy revealed a putrescent cervical cancer. To stop the bleeding the catheter 100 was inserted into a vaginal and uterine cavity, and a tumor area was isolated by the balloons of the catheter 100. The instillation with a hemostatic drug was performed for the tumor through the functional channel of the catheter 100. The bleeding was stopped, and the patient left the hospital.
Example No. 19. Patient #19 underwent surgery to restore the colon continuity. On day 10, anastomotic leakage was detected. To treat the patient, catheter 100 was inserted endoscopically through the rectum so that the anastomotic area with the defect was isolated from the proximal and distal parts of the large intestine after the balloon inflation. In addition, the channels for balloon inflation and manipulation were led out through the rectum. The balloons were inflated with air—100 ml, the manipulation channel was connected to a controlled negative pressure system with lavage function. The mode of 20 minutes of alternating negative pressure (50 to 100 mm Hg) with a subsequent injection of 10.0 ml of aqueous antiseptic solution (chlorohexidine) was set. A drainage was placed in the dehiscence area in the abdominal cavity to control the presence of intestinal discharge. The patient was treated and transferred to a fiber-free diet and nutrition was fed enterally. The treatment lasted for 6 weeks and consisted of endoscopic change of the catheter 100 once a week. At control imaging, the area was cleared of fibrin, fresh granulation appeared, and the defect was closed, and there was no deformation or stenosis of the anastomosis area due to the frame property of catheter 100. During the entire period of treatment, the patient had gas and regular loose stools.
Example No. 20. Patient #20, suffering from stage IV esophageal cancer, was admitted to the hospital due to complications caused by the tumor: esophageal-tracheal fistula formation and bleeding from the tumor. Complications were detected during endoscopy, and the cancerous stenosis of the esophagus did not negatively impact the endoscopy procedure. Endoscopic methods of hemostasis were ineffective, vascular embolization was impossible due to segmental type of blood supply to the esophagus, surgical intervention was not possible because of the somatic status of the patient and unresectable tumor. For treatment, catheter 100 including net member 16 was placed so that the bleeding tumor with fistula was located between the balloons. The manipulation channel was connected to a vacuum therapy system with instillation function, the following parameters were set: alternating negative pressure for 20 minutes (50 to 100 mm Hg) with a subsequent injection of 20.0 ml of hemostatic solution. As a result, the fistula was disconnected, and the bleeding was stopped by both medications and additional mechanical pressure of the stretched net member 16. Due to the frame function of the stent, the lumen of the esophagus was dilated, and the patient was able to take liquid and puréed food through the mouth on his own. Due to negative pressure, blood was not allowed to enter the airways through the fistula. When removing catheter 100 outfitted with net member 16, the balloons were maximally deflated (deflated with the aid of the net member 16): the diameter of catheter 100 decreased and tumor traumatization was avoided during its removal. After removal of catheter 100 outfitted with net member 16, an esophageal covered stent was placed in the esophageal cavity.
Example No. 21. Patient #21 with diagnosed decompensated carotid arteries stenosis and demonstrated clinical signs of transient cerebrovascular disorder. The patient was examined and consulted by a neurologist and vascular surgeon; replacement of carotid arteries was recommended to improve the cerebral circulation. Existing 90% occlusion increased a risk of ischemic stroke occurrence during surgical approach. For preventive purposes, catheter 100 was placed to both maintain blood flow and to minimize blood loss during artery mobilization. The catheter 100 was inserted through the femoral artery and positioned before the surgery. Carotid artery plastic surgery was performed without any accompanied blood loss. The patient had no cerebrovascular disorders in the postoperative period and no further complications were observed.
Example No. 22. Patient #22 was admitted to the regional hospital with an abdominal aorta injury. The patient was moved to the operating room and during laparotomy the extended defect of aorta was verified. For further medical decision (suturing, replacement) vascular surgeon consultation was required. Vascular surgeon in charge was invited to the patient due to the persisting bleeding that was temporarily stopped by means of physical pressing and clipping with artery forceps. The assisting surgeon executed femoral access, and catheter 100 was inserted retrogradely through the femoral artery and positioned in such a way that the injury was located between the balloons that were later inflated. The bleeding was stopped with the peripheral blood flow maintenance below the injury site. The vascular surgeon was delivered by the ambulance team 30 minutes later, and it was decided to suture the injury. The patient had no ischemic or necrotic changes in distal organs and tissues in the postoperative period.
Example No. 23. Patient #23 was admitted to the hospital with a severe headache and neurological symptoms. Examination revealed dissection of right medial cerebral artery aneurysm. The patient was transferred to X-ray operating room, and catheter 100 was inserted for treatment purposes. Balloons were inflated in such a way that the aneurysm entrance was isolated. An adhesive gel was injected in the aneurysm cavity via the manipulation channel (e.g., functional channel 3), and 30-minute exposure period ensured the adhesive fixation to prevent its leak and embolization of the distal part of arteries by the gel. The patient received treatment in the postoperative period without any complications observed.
Example No. 24. In combat conditions, Patient #24 received a bullet wound at the level of the middle third of the right thigh accompanied by active persisting bleeding. A pressing hemostatic bandage was applied, and the patient was admitted to a medical unit. During a surgical approach, proximal and distal parts of the injured artery were identified. A defect between the proximal and distal parts of the artery was identified to be about 5 cm long, requiring a placement of a vessel graft, which was impossible in this medical unit. Urgent relocation of the patient into the nearest specialized vascular center was necessary. Transportation time was about 2 hours, which carried a risk of limb loss. To restore a peripheral perfusion, from the proximal part of the artery, catheter 100 was inserted and advanced to the efferent end of the artery. The upper balloon occluded and fixated the proximal part of the artery, while the lower balloon did that for the distal part. As a result of this manipulation, peripheral blood circulation was restored, which was verified by the presence of pulse in the distal artery. A sterile bandage was applied, the limb was immobilized, and the patient was urgently moved to the nearest vascular center. The application of catheter 100 had stabilized the patient's limb for a long enough time to facilitate transportation to the specialized vascular center, which subsequently prevented lower limb amputation.

Therefore, the above-described catheter 100, catheter 200, and/or catheter 300 allows for conducting or performing functional investigation of any hollow organ, and also for providing appropriate treatments. It should be noted that catheter 300 may have been used in place of catheter 100 with similar effect as described in Examples 12, 14, 15, 18, 19, 20, 21, 22, 23, and 24. Furthermore, examination and/or laboratory analysis of biological liquid, collected by using the catheter 100, catheter 200, and/or catheter 300, allows one with high accuracy to differentially diagnose inflammatory formations, benign formations and malignant growth, and also infectious diseases. By evacuating specific liquid and/or by supplying a liquid or gaseous medium to the isolated interior of a hollow organ the catheter 100, catheter 200, and/or catheter 300 allows for the treatment of inflammatory diseases, bleeding, defects of walls of hollow organs and treatment of diseases of organs connected to hollow organs. Producing of an isolated area with a function of bypass during surgical manipulation allows one to control hemostasis, and also to perform surgical manipulations, in particular resection of a site of a hollow organ, such as a vessel, with its subsequent plasty. It is to note further that the catheter 100, catheter 200, and/or catheter 300 according to the present invention or the above-described system for isolating a region in a hollow organ of a mammal (not shown), which may include the catheter 100, catheter 200, and/or catheter 300, provides capability to collect high volume of biological material in a normal physiological and biochemical state without additional stimulation of excretory function of a hollow organ or an organ connected with a hollow organ, in particular due to the creation in the isolated area of a hollow organ, into the lumen of which the catheter 100, catheter 200, and/or catheter 300 is inserted, of a negative pressure corresponding to physiological negative pressure which may be produced, for example, due to the peristalsis of intestine.

What is claimed is:

1. A catheter for isolating a region in a mammal hollow organ, comprising:
an elongate body comprising a proximal end, a distal end, and a central lumen disposed within the elongate body, the elongate body defining a longitudinal axis, both the proximal end and the distal end of the elongate body configured to be inserted into a lumen of the mammal hollow organ, the proximal end defining a first opening of the elongate body and the distal end defining a second opening of the elongate body;
a plurality of proximal extensions coupled to the proximal end of the elongate body, the plurality of proximal extensions comprising a plurality of individual channels that are continuous with the elongate body such that the plurality of individual channels are embedded within the elongate body;
two balloons, individually and separately disposed along the elongate body and configured to be inflated to isolate an interior of the hollow organ therebetween;
a functional channel of the plurality of individual channels comprising one or more functional openings provided in the elongate body between the two balloons, wherein the functional channel is designed to allow:
a negative pressure to be produced in the isolated interior to take a fluid or gaseous medium therefrom; and/or
a liquid or gaseous medium to be supplied into the isolated interior; and
a second channel of the plurality of individual channels, the second channel extending in the elongate body to deliver a fluid or gaseous medium to at least one of the two balloons to provide inflation thereof.

2. The catheter of claim 1, wherein, the first opening comprises an oblique opening within the elongate body comprising an angle between approximately 15 degrees to approximately 65 degrees with respect to the longitudinal axis defined by the elongate body.

3. The catheter of claims 1 or 2, wherein the second opening comprises an oblique opening within the elongate body comprising an angle between approximately 15 degrees to approximately 65 degrees with respect to the longitudinal axis defined by the elongate body.

4. The catheter of any one of claims 1-3, wherein the central lumen is configured to facilitate bidirectional flow through the first opening and the second opening.

5. The catheter of any one of claims 1-4, wherein the second opening further comprises an opening at the distal end configured to facilitate insertion of the elongate body into the lumen of the mammal hollow organ.

6. The catheter of any one of claims 1-5, wherein each of the plurality of channels are hermetically isolated from each other and from the central lumen.

7. The catheter of any one of claims 1-6, further comprising two enclosing projections on the elongate body, wherein the one or more functional openings are positioned between the two enclosing projections.

8. The catheter of claim 7, wherein the enclosing projections are ring-shaped and positioned adjacent to or against the one or more functional openings.

9. The catheter of any one of claims 1-8, wherein the elongate body further comprises a net member designed such that it at least partly encloses the catheter part defined by the balloons and covers the one or more functional openings.

10. The catheter of claim 9, wherein the net member is attached to the balloons and/or covers the balloons such that the net member become strained when the balloons are inflated.

11. The catheter of any one of claims 1-10, wherein the net member further comprises a compression member integrated into the net member that is configured to provide a compressive force on the balloons when the compression member is pulled proximally, thereby facilitating deflation of the balloons.

12. The catheter of any one of claims 1-10, wherein the net member further comprises a compression member affixed to a proximal end of the net member, the compression member configured to elongate the net member when the compression member is pulled proximally, thereby reducing a diameter of the net member and facilitating deflation of the balloons.

13. A system for isolating a region in a hollow organ of a mammal, comprising:
inserting the catheter of any one of claims 1-12; and
a functional device connected to the functional channel to allow the fluid or gaseous medium to be evacuated therefrom or a liquid or gaseous medium to be supplied thereto.

14. A method of isolating a region in a hollow organ of a mammal, comprising:
inserting the catheter of any one of claims 1-12 into a lumen of the mammal hollow organ;
inflating the balloons to isolate an interior of the mammal hollow organ therebetween;
delivering through the functional channel and the one or more functional openings of the catheter a negative pressure in the isolate interior for taking therefrom a fluid or a gaseous medium being specific to the hollow organs or organs connected to the hollow organs, or supplying, by means of the functional channel and the one or more functional openings, a liquid or gaseous medium to the isolated interior.

15. The use of the catheter of any one of claims 1-12 for diagnosing or monitoring the development or monitoring the treatment of at least one disease selected from a group comprising: bleeding, haemostasis, gastritis, acute and chronic pancreatitis, autoimmune pancreatitis pancreatic neoplasms and cancer, bile duct cancer, cholangiocarcinoma, hepatocellular cancer, cholangitis, cholelithiasis, defects of a hollow organ wall, fistula of hollow organs, autoimmune hepatitis, infectious hepatitis, sclerotic cholangitis, Crohn disease, ulcerative colitis, eosinophilic esophagitis, aneurismal or diverticular protrusions of a hollow organ wall, bleeding of hollow organs, bleeding into hollow organs, strictures of hollow organs, esophagus neuromuscular disorders, stomach neuromuscular disorders, duodenum neuromuscular disorders, neuromuscular disorders of the small intestine and colon and rectum, neuromuscular disorders of bile ducts, neuromuscular disorders of urinoexcretory ways, and neuromuscular disorders of respiratory tracts, vascular thrombosis and embolization, vessels' malformations.

16. The use of the catheter of any one of claims 1-12 for treatment of at least one disease selected from a group comprising: bleeding, pancreatitis, cholangitis, gastrointestinal wall defects, fistulas of hollow organs, vascular defects, vascular aneurisms, vascular thrombosis and embolization, defects of a hollow organ wall, bleeding of hollow organs, bleeding into hollow organs, gastrointestinal hemorrhages, bleeding from the windpipe (trachea), bronchial bleeding, pulmonary hemorrhage, uterine bleeding, vessels' malformations, Crohn disease, ulcerative colitis, eosinophilic esophagitis, malignant diseases that occur in hollow organs and organs that are connected to hollow organs.

17. The use of the catheter of any one of claims 1-12 to isolate an area of a hollow organ when performing a surgical procedure or performing surgical manipulations.
